(19) United States
(12) Patent Application Publication
(10) Pub. No.: US 2025/0064631 A1
(43) Pub. Date: Feb. 27, 2025

(54) PATIENT TEMPERATURE MANAGEMENT SYSTEM

(71) Applicant: Stryker Corporation, Kalamazoo, MI (US)

(72) Inventors: Marco Constant, Portage, MI (US); Christopher John Hopper, Kalamazoo, MI (US); Caleb D. Gossens, Ravenna, MI (US); Michael J. Estes, Portage, MI (US); Hannah A. Porter, Portage, MI (US); Daniel P. DesRosiers, Decatur, MI (US)

(21) Appl. No.: 18/724,938

(22) PCT Filed: Mar. 17, 2023
(86) PCT No.: PCT/US2023/015522

§ 371 Date: Jun. 27, 2024

Related U.S. Application Data
(60) Provisional application No. 63/321,066, filed on Mar. 17, 2022.

(51) Int. Cl. A61F 7/00

(52) U.S. Cl. CPC A61F 7/0085; A61F 2007/0054; A61F 2007/0095

(57) ABSTRACT

A thermal control system for controlling a patient's temperature includes a thermal control unit and an off-board computing device, such as a server. The thermal control unit includes a fluid inlet, a fluid outlet, a pump, a heat exchanger, a controller, and a transceiver for communicating with the server. The server is adapted to communicate with a device and cause the device to perform one or more of the following: display real-time temperature data on a graph with adjacent horizontal bars whose length correspond to an amount of time an alarm existed; display segments of a graph corresponding to the time around which an alarm occurred: display different sets of data according to the role of a user of the device: display thermal therapy data for only selected thermal therapy sessions that are associated with the particular user of the device; and display thermal therapy data simultaneously with bed data.

### CROSS-REFERENCE TO RELATED APPLICATION

This application claims priority to U.S. provisional patent application Ser. No. 63/321,066 filed Mar. 17, 2022, by inventors Marco Constant et al. and entitled THERMAL CONTROL SYSTEMS WITH NETWORK COMMUNICATION, the complete disclosure of which is incorporated herein by reference.

### BACKGROUND

The present disclosure relates to a thermal control system for controlling the temperature of circulating fluid that is delivered to one or more thermal devices positioned in contact with a patient.

Thermal control systems are known in the art for controlling the temperature of a patient by providing a thermal control unit that supplies temperature-controlled fluid to one or more thermal pads or catheters positioned in contact with a patient. The thermal control unit includes one or more heat exchangers for controlling the temperature of the fluid and a pump that pumps the temperature-controlled fluid to the pad(s) and/or catheter. After passing through the pad(s) and/or catheter, the fluid is returned to the thermal control unit where any necessary adjustments to the temperature of the returning fluid are made before being pumped back to the pad(s) and/or catheter. In some instances, the temperature of the fluid is controlled to a static target temperature, while in other instances the temperature of the fluid is varied as necessary in order to automatically effectuate a target patient temperature.

Thermal control units typically include a control panel adapted to allow the user to input information for using the thermal control unit, as well as for displaying information useful to the user of the thermal control unit. The control panel also enables the user to execute one or more functions of the patient support apparatus, such as, but not limited to, inputting a target patient temperature; choosing a cooling rate; choosing a warming rate; defining a cooling, warming, and/or hold time; determining which alarms to implement; selecting alarm characteristics; controlling what information is displayed, recorded, and/or transmitted off-board the thermal control unit; choosing what sensor inputs are to be used during the thermal therapy session, etc.

### SUMMARY

A thermal control system according various aspects of the present disclosure includes a server adapted to communicate with or more thermal control units, one or more user devices (e.g. smart phones, tablet computers, etc.), and, in some aspects, one or more patient support apparatuses. The server is adapted to allow data from the thermal control unit(s) to be viewed and/or processed by the user devices, and/or to allow the thermal control unit(s) to be remotely controlled by the user devices. In some aspects, the server is adapted to provide access to the remote controlling, viewing, and/or processing via a web application, such as a web browser, that is installed on the user devices. The server may also, or alternatively, be adapted to communicate with native software applications that are written for specific operating systems of the user devices (e.g. the Android, IOS, etc.).

According to a first aspect of the present disclosure, a thermal control system for controlling a patient's temperature during a thermal therapy session is provided. The thermal control system includes a thermal control unit and a first server. The thermal control unit includes a circulation channel, a pump, a heat exchanger, a temperature sensor, a controller, and a transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature, as well as to detect an alarm during the thermal therapy session. The transceiver is adapted to transmit the alarm and readings from the temperature sensor to the first server. The first server is adapted to communicate with a device having a display, to cause the device to display an alarm indicator on the display and, in response to a user selecting the alarm indicator, to automatically display a graph of the temperature readings from the temperature sensor over a time period. The time period includes a first time segment and a second time segment, and the first time segment includes time prior to commencement of the alarm and the second time segment includes time after the commencement of the alarm.

According to other aspects of the present disclosure, the time period may have a fixed duration.

According to another aspect of the present disclosure, a thermal control system for controlling a patient's temperature during a thermal therapy session is provided. The thermal control system includes a thermal control unit and a first server. The thermal control unit includes a circulation channel, a pump, a heat exchanger, a temperature sensor, a controller, and a transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature. The transceiver is adapted to communicate with the first server. The first server is adapted to communicate with a second server and with a device associated with a user, to receive a user ID from the device, to consult the second server to determine a role of the user, to share a first set of data with the device if the user has a first role, and to share a second set of data with the device if the user has a second role different from the first role.

In some aspects, the second server is an electronic medical records server, while in other aspects the second server is a non-electronic medical records server.

According to another aspect of the present disclosure, a thermal control system for controlling a patient's temperature during a thermal therapy session is provided. The thermal control system includes a plurality of thermal controls unit and a first server. Each of the thermal control units includes a circulation channel, a pump, a heat exchanger, a temperature sensor, a controller, and a transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature, as well as to assign a thermal therapy session ID to each thermal therapy session performed by the thermal control unit. The transceiver is adapted to communicate the thermal therapy session ID to the first server. The first server is adapted to receive a user ID from a first user device, to determine a first set of thermal therapy sessions associated with the user ID, to determine a second set of thermal therapy sessions not associated with the user ID, to forward data regarding the first set of thermal therapy sessions to the device, and to not forward data regarding the second set of thermal therapy sessions to the device.

According to another aspect of the present disclosure, a thermal control system for controlling a patient's temperature during a thermal therapy session is provided. The thermal control system includes a thermal control unit and a first server. The thermal control unit includes a circulation channel, a pump, a heat exchanger, a temperature sensor, a controller, and a transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature. The first server is adapted to receive a command from a user device associated with a user of the thermal control unit and to forward the command to the thermal control unit. The transceiver of the thermal control unit is adapted to receive the command and the controller is adapted to adapted to implement the command.

According to some aspects, the command is a command is to mute an alarm on the thermal control unit.

In some aspects, the command is a command to add a new thermal therapy session option to the thermal control unit, and the controller is adapted to display the new thermal therapy session option on a display of the thermal control unit.

In some aspects, the command is a command to install new software on the thermal control unit.

In some aspects, the command is a command to pause a currently active thermal therapy session.

According to another aspect of the present disclosure, a thermal control system for controlling a patient's temperature during a thermal therapy session is provided. The thermal control system includes a plurality of thermal controls unit and a first server. Each of the thermal control units includes a circulation channel, a pump, a heat exchanger, a temperature sensor, a controller, and a transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature, as well as to assign a thermal therapy session ID to each thermal therapy session performed by the thermal control unit. The transceiver is adapted to communicate the thermal therapy session ID to the first server. The first server is adapted to communicate with a device associated with a user of the thermal therapy unit, and to cause the device to display a selection option and a comparison option. The selection option is adapted to enable the user to select a first thermal therapy session and a second thermal therapy session, and the comparison option is adapted to cause the device to compare data from the first thermal therapy session to data from the second thermal therapy session.

In some aspects, the first server is further adapted to cause the user device to display an average of a first parameter from the first thermal therapy session and a second parameter from the second thermal therapy session.

According to still another aspect of the present disclosure, a thermal control system for controlling a patient's temperature during a thermal therapy session is provided. The thermal control system includes a plurality of thermal controls unit, a plurality of patient support apparatuses, and a first server. Each of the thermal control units includes a circulation channel, a pump, a heat exchanger, a temperature sensor, a controller, and a first transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature. The first transceiver is adapted to communicate thermal therapy data to the first server. Each of the patient support apparatuses include a frame, a support surface adapted to support a patient thereon, and a second transceiver adapted to communicate bed status data to the first server. The first server is adapted to receive the thermal therapy data and the bed status data, to forward the thermal therapy data and the bed status data to a first device having a display, and to cause the first device to simultaneously display the thermal therapy data and the bed status data.

In some aspects, the first server is adapted to cause the first device to arrange the thermal therapy data and bed status data on the display according to rooms within a healthcare facility.

In some aspects, the first server is adapted to display additional patient support apparatus data and/or thermal therapy data on the display in response to a user selecting a particular room.

According to still another aspect of the present disclosure, a thermal control system for controlling a patient's temperature during a thermal therapy session is provided. The thermal control system includes a thermal control unit and a first server. The thermal control unit includes a circulation channel, a pump, a heat exchanger, a temperature sensor, a controller, and a transceiver. The circulation channel is coupled to a fluid inlet and a fluid outlet. The pump is adapted to circulate fluid through the circulation channel from the fluid inlet to the fluid outlet. The heat exchanger is adapted to add or remove heat from the fluid circulating in the circulation channel. The temperature sensor is adapted to detect a temperature of the fluid. The controller is adapted to control the heat exchanger in order to control the patient's temperature. The first server is adapted to communicate with a user device having a display, to cause the user device to display real time temperature readings from the temperature sensor on a graph having a horizontal time axis and a vertical temperature axis, and to cause the user device to display horizontal bars adjacent to the graph. The horizontal bars correspond to alarms and have lengths proportional to the amount of time the alarms have lasted.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration is used in the description herein of various embodiments (e.g. first, second, third, etc.). Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus and a thermal control unit for controlling the temperature of a patient supported on the patient support apparatus;

FIG. 2 is a perspective view of the thermal control unit of FIG. 1;

FIG. 3 is a block diagram of a thermal control system that includes the thermal control unit, the patient support apparatus, and one or more devices in communication with a healthcare facility network;

FIG. 4 is a plan view of a control panel of the thermal control unit showing a therapy mode selection screen displayed thereon;

FIG. 5 is a plan view of the control panel of the thermal control unit showing a therapy preset selection screen displayed thereon;

FIG. 6 is a plan view of the control panel of the thermal control unit showing a summary screen that may be displayed during an in-progress therapy session;

FIG. 7 is an example of a login screen that may be displayed on a user device adapted to communicate with the thermal control system;

FIG. 8 is an example of an active case overview screen that may be displayed on the user device;

FIG. 9 is an example of the active case overview screen of FIG. 8 illustrating an alarm associated with one of the active cases;

FIG. 10 is an example of an active case overview screen that may be displayed on the user device;

FIG. 11 is a close up view of a graph that may be displayed on the active case overview screen that includes an alarm data window and a note window;

FIG. 12 is an example of an alarm overview screen that may be displayed on the user device;

FIG. 13 is an example of the alarm overview screen of FIG. 12 showing additional information about a selected alarm;

FIG. 14 is an example of a note overview screen that may be displayed on the user device;

FIG. 15 is an example of a thermal therapy session selection screen that may be displayed on the user device for selecting a particular thermal therapy session to be associated with a particular patient;

FIG. 16 is an example of a patient selection screen that may be displayed on the user device for selecting a particular patient to be associated with a particular thermal therapy session;

FIG. 17 is an example of a first remote control screen that may be displayed on the user device for remotely controlling a warming phase carried out by the thermal control unit;

FIG. 18 is an example of a second remote control screen that may be displayed on the user device for remotely controlling a cooling phase carried out by the thermal control unit;

FIG. 19 is an example of a first status screen that may be displayed on the user device indicating that the remote control settings of FIGS. 17 and/or 18 are being sent to the thermal control unit;

FIG. 20 is an example of a second status screen that may be displayed on the user device indicating that the remote control settings of FIGS. 17 and/or 18 were successfully received by the thermal control unit;

FIG. 21 is an example of a third status screen that may be displayed on the user device indicating that the remote control settings of FIGS. 17 and/or 18 were not successfully received by the thermal control unit;

FIG. 22 is an example of a post-therapy overview screen that may be displayed on the user device and that illustrates a plurality of filtered cases and a plurality of unfiltered cases;

FIG. 23 is the post-therapy overview screen of FIG. 22 shown with a filter window displayed in an expanded form;

FIG. 24 is an example of a filter creation screen that may be displayed on the user device in order to allow a user to create new filter criteria for filtering thermal therapy cases;

FIG. 25 is an example of a post-therapy individual case screen that may be displayed on the user device and that indicates details regarding a selected thermal therapy case;

FIG. 26 is the post-therapy individual case screen of FIG. 25 showing further details regarding a maintenance phase that the user has selected;

FIG. 27 is an example of a report configuration screen that may be displayed on the user device and that allows the user to group thermal therapy cases together to create a report;

FIG. 28 is an example of a comparison screen that may be displayed on the user device and that compares two different thermal therapy cases together in a side-by-side fashion;

FIG. 29 is an example of an alarm selection screen that may be displayed on the user device and that allows the user to select different alarms that are to apply to the thermal control unit;

FIG. 30 is an example of an alarm configuration screen that may be displayed on the user device and that allows the user to configure characteristics of individual alarms;

FIG. 31 is an example of a therapy preset overview screen that may be displayed on the user device and that indicates which therapy presets are defined for usage by the thermal control unit;

FIG. 32 is an example of a therapy preset definition screen that may be displayed on the user device and that allows the user to define a new therapy preset to be followed by the thermal control unit;

FIG. 33 is an example of a role permission screen that may be displayed on the user device and that allows an authorized administrator to control what features of the thermal control system are available to users based on roles assigned to the users;

FIG. 34 is an example of a first linking screen that may be displayed on the user device and that allows a user to link a particular patient to a particular thermal therapy session;

FIG. 35 is an example of a second linking screen that may be displayed on the user device after displaying the screen of FIG. 34 and that allows a user to link a particular patient to a particular thermal therapy session;

FIG. 36 is an example of a third linking screen that may be displayed on the user device after displaying the screen of FIG. 35 and that allows a user to link a particular patient to a particular thermal therapy session;

FIG. 37 is an example of a fourth linking screen that may be displayed on the user device after displaying the screen of FIG. 36 and that allows a user to link a particular patient to a particular thermal therapy session;

FIG. 38 is an example of a fifth linking screen that may be displayed on the user device after displaying the screen of FIG. 37 and that allows a user to link a particular patient to a particular thermal therapy session;

FIG. 39 is an example of a sixth linking screen that may be displayed on the user device after displaying the screen of FIG. 38 and that allows a user to link a particular patient to a particular thermal therapy session;

FIG. 40 is an example of a seventh linking screen that may be displayed on the user device after displaying the screen of FIG. 39 and that allows a user to link a particular patient to a particular thermal therapy session;

FIG. 41 is an example of an eighth linking screen that may be displayed on the user device after displaying the screen of FIG. 40 and that indicates that the process of linking a particular patient to a particular thermal therapy session is taking place;

FIG. 42 is an example of a ninth linking screen that may be displayed on the user device after displaying the screen of FIG. 41 and that indicates that the process of linking a particular patient to a particular thermal therapy session has been successful;

FIG. 43 is an example of a tenth linking screen that may be displayed on the user device after displaying the screen of FIG. 42 and that indicates that the process of linking a particular patient to a particular thermal therapy session has not been successful;

FIG. 44 is an example of a combined dashboard for displaying patient support apparatus data and thermal control system data;

FIG. 45 is a plan view of the control panel of the thermal control unit showing a therapy session screen displayed thereon;

FIG. 46 is a plan view of the control panel of the thermal control unit showing a modified therapy preset selection screen, similar to what is shown in FIG. 5, displayed thereon;

FIG. 47 is a plan view of the control panel of the thermal control unit showing a screen saver screen for an in-progress therapy session displayed thereon;

FIG. 48 is a plan view of a portion of the control panel of the thermal control unit showing a viewing options screen in which a “count down” option and a “minutes” option have been selected;

FIG. 49 is a plan view of a portion of the control panel of the thermal control unit showing the viewing options screen of FIG. 48, but with a “count up” option and an “hours” option selected;

FIG. 50 is a plan view of a portion of the control panel showing several controls, including a power control, as they are displayed when no therapy session is currently in progress;

FIG. 51 is a plan view of the same portion of the control panel of FIG. 50 showing the power control as it may be displayed when a therapy session is currently in progress; and

FIG. 52 is a plan view of a portion of the control panel of the thermal control unit showing an alarm history screen.

### DETAILED DESCRIPTION

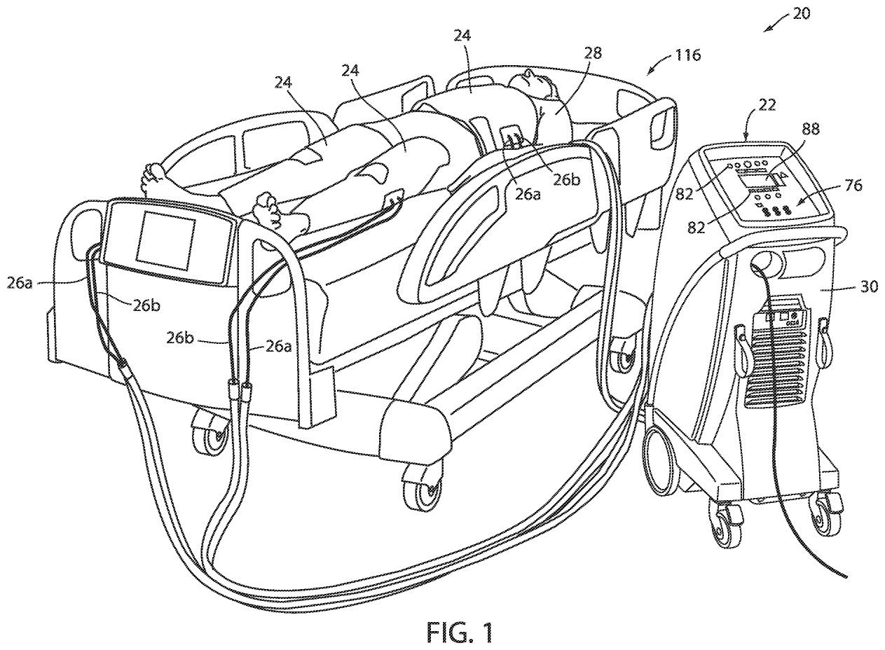

A portion of a thermal control system 20 according to one embodiment of the present disclosure is shown in FIG. 1. Thermal control system 20 is adapted to control the temperature of a patient 28, which may involve raising, lowering, and/or maintaining the patient's temperature. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, wraps, vests, patches, caps, catheters, or other structures that receive temperature-controlled fluid. For purposes of the following written description, thermal therapy devices 24 will be referred to as thermal wraps 24, but it will be understood by those skilled in the art that this terminology is used merely for convenience and that the phrase “thermal wrap” is intended to cover all of the different variations of thermal therapy devices 24 mentioned above (e.g. blankets, vests, patches, caps, catheters, etc.) and variations thereof.

Thermal control unit 22 is coupled to thermal wraps 24 via a plurality of hoses 26. Thermal control unit 22 delivers temperature-controlled fluid (such as, but not limited to, water or a water mixture) to the thermal wraps 24 via the fluid supply hoses 26a. After the temperature-controlled fluid has passed through thermal wraps 24, thermal control unit 22 receives the temperature-controlled fluid back from thermal wraps 24 via the return hoses 26b.

In the portion of thermal control system 20 shown in FIG. 1, three thermal wraps 24 are used in the treatment of patient 28. A first thermal wrap 24 is wrapped around a patient's torso, while second and third thermal wraps 24 are wrapped, respectively, around the patient's right and left legs. Other configurations can be used and different numbers of thermal wraps 24 may be used with thermal control unit 22, depending upon the number of inlet and outlet ports that are included with thermal control unit 22. By controlling the temperature of the fluid delivered to thermal wraps 24 via supply hoses 26a, the temperature of the patient 28 can be controlled via the close contact of the wraps 24 with the patient 28 and the resultant heat transfer therebetween.

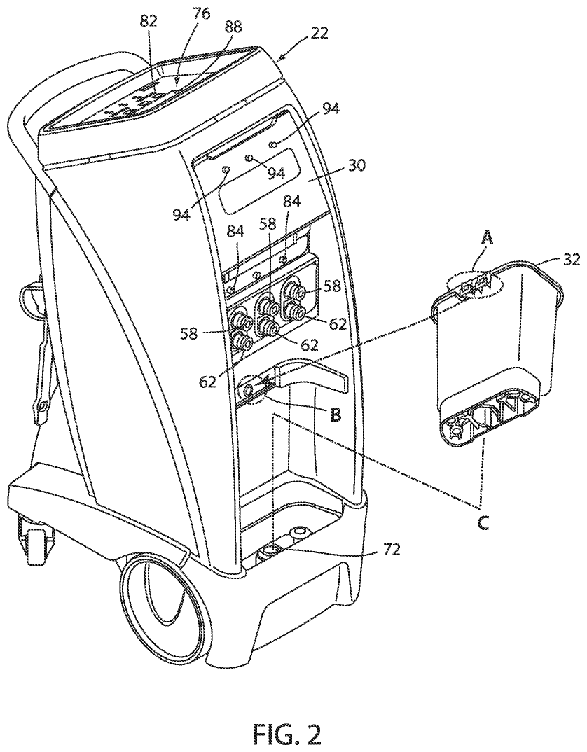

As shown more clearly in FIG. 2, thermal control unit 22 includes a main body 30 to which a removable reservoir 32 may be coupled and uncoupled. Removable reservoir 32 is configured to hold the fluid that is to be circulated through thermal control unit 22 and the one or more thermal wraps 24. By being removable from thermal control unit 22, reservoir 32 can be easily carried to a sink or faucet for filling and/or dumping of the water or other fluid. This allows users of thermal control system 20 to more easily fill thermal control unit 22 prior to its use, as well as to drain thermal control unit 22 after use.

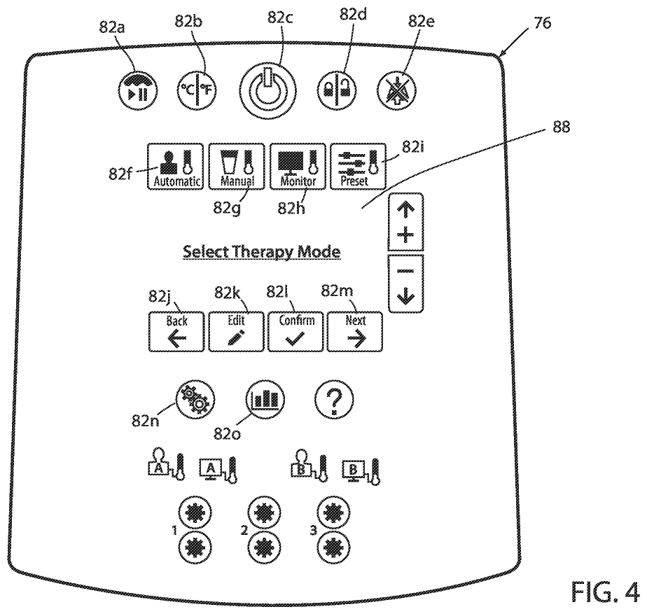

As can also be seen in FIG. 2, thermal control unit 22 includes a plurality of outlet ports 58 (three in the particular example of FIG. 2), a plurality of inlet ports 62 (three in this particular example). Outlet ports 58 are adapted to fluidly couple to supply hoses 26a and inlet ports are adapted to fluidly couple to return hoses 26b. Thermal control unit 22 also includes a plurality of patient temperature probe ports 84, a plurality of auxiliary ports 94, and a control panel 76 having a plurality of dedicated controls 82 and a display 88 (see also FIGS. 4-6). The patient temperature probe ports 84, auxiliary ports 94, and control panel 76 are described in more detail below. In some embodiments, auxiliary ports 94 are omitted.

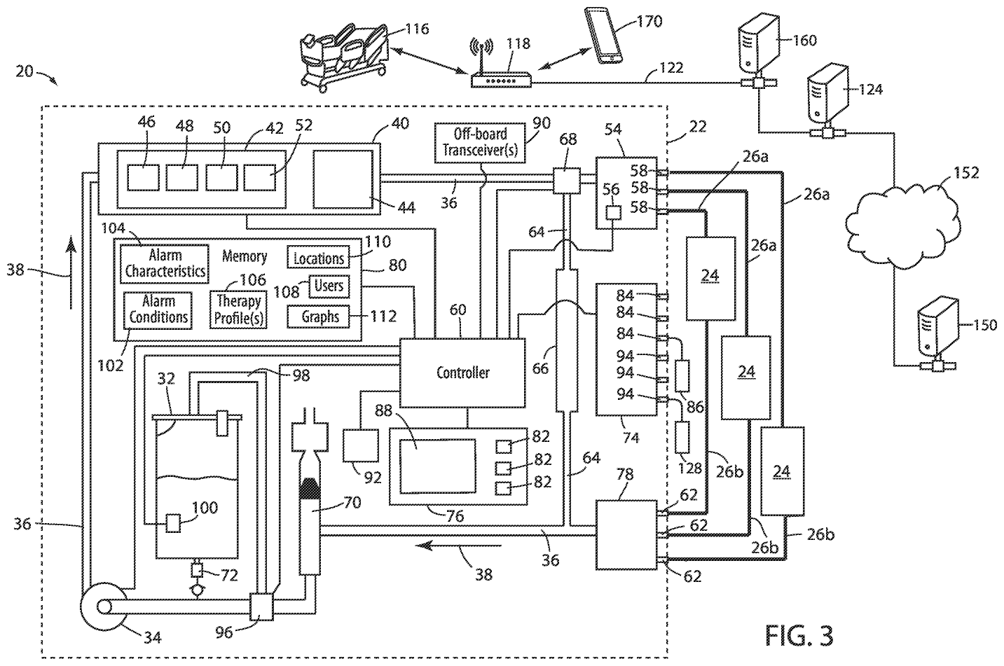

As shown in FIG. 3, thermal control unit 22 includes a pump 34 for circulating fluid through a circulation channel 36. Pump 34, when activated, circulates the fluid through circulation channel 36 in the direction of arrows 38 (clockwise in FIG. 3). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In the embodiment shown in FIG. 3, heat exchanger 40 includes a chiller 42 and a heater 44. Further, in the embodiment shown in FIG. 3, chiller 42 is a conventional vapor-compression refrigeration unit having a compressor 46, a condenser 48, an evaporator 50, an expansion valve (not shown), and a fan 52 for removing heat from the compressor 46. Heater 44 is a conventional electrical resistance-based heater. Other types of chillers and/or heaters may be used.

After passing through heat exchanger 40, the circulating fluid is delivered to an outlet manifold 54 having an outlet temperature sensor 56 and a plurality of outlet ports 58. Temperature sensor 56 is adapted to detect a temperature of the fluid inside of outlet manifold 54 and report it to a controller 60. Outlet ports 58 are coupled to supply hoses 26a. Supply hoses 26a are coupled, in turn, to thermal wraps 24 and deliver temperature-controlled fluid to the thermal wraps 24. The temperature-controlled fluid, after passing through the thermal wraps 24, is returned to thermal control unit 22 via return hoses 26b. Return hoses 26b couple to a plurality of inlet ports 62. Inlet ports 62 are fluidly coupled to an inlet manifold 78 inside of thermal control unit 22.

Thermal control unit 22 also includes a bypass line 64 fluidly coupled to outlet manifold 54 and inlet manifold 78 (FIG. 3). Bypass line 64 allows fluid to circulate through circulation channel 36 even in the absence of any thermal wraps 24 or hoses 26a being coupled to any of outlet ports 58. In the illustrated embodiment, bypass line 64 includes a filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both. In some embodiments, filter 66 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 64 is controllable by way of a bypass valve 68 positioned at the intersection of bypass line 64 and outlet manifold 54 (FIG. 3). When open, bypass valve 68 allows fluid to flow through circulation channel 36 to outlet manifold 54, and from outlet manifold 54 to the connected thermal wraps 24. When closed, bypass valve 68 stops fluid from flowing to outlet manifold 54 (and thermal wraps 24) and instead diverts the fluid flow along bypass line 64. In some embodiments, bypass valve 68 may be controllable such that selective portions of the fluid are directed to outlet manifold 54 and along bypass line 64. In some embodiments, bypass valve 68 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference. In other embodiments, bypass valve 68 may be a pressure operated valve that allows fluid to flow along bypass line 64 if the fluid pressure in circulation channel 36 exceeds the cracking pressure of the bypass valve 68. Still further, in some embodiments, bypass valve 68 may be omitted and fluid may be allowed to flow through both bypass line 64 and into outlet manifold 54.

The incoming fluid flowing into inlet manifold 78 from inlet ports 62 and/or bypass line 64 travels back toward pump 34 and into an air remover 70. Air remover 70 includes any structure in which the flow of fluid slows down sufficiently to allow air bubbles contained within the circulating fluid to float upwardly and escape to the ambient surroundings. In some embodiments, air remover 70 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 70, the circulating fluid flows past a valve 72 positioned beneath fluid reservoir 32. Fluid reservoir 32 supplies fluid to thermal control unit 22 and circulation channel 36 via valve 72, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 72, the circulating fluid travels to pump 34 and the fluid circuit is repeated.

Controller 60 of thermal control unit 22 is contained within main body 30 of thermal control unit 22 and is in electrical communication with pump 34, heat exchanger 40, outlet temperature sensor 56, bypass valve 68, a sensor module 74, control panel 76, a memory 80, one or more transceivers 90, and, in some embodiments, one or more other sensors, such as, but not limited to, a location sensor 92. Controller 60 includes any and all electrical circuitry and components necessary to carry out the functions and algorithms described herein, as would be known to one of ordinary skill in the art. Generally speaking, controller 60 may include one or more microcontrollers, microprocessors, and/or other programmable electronics that are programmed to carry out the functions described herein. It will be understood that controller 60 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

Control panel 76 allows a user to operate thermal control unit 22. Control panel 76 communicates with controller 60 and includes a display 88 and a plurality of dedicated controls 82a, 82b, 82c, etc. Display 88 may be implemented as a touch screen, or, in other embodiments, as a non-touch-sensitive display. Dedicated controls 82 may be implemented as buttons, switches, dials, or other dedicated structures. In any of the embodiments, one or more of the functions carried out by a dedicated control 82 may be replaced or supplemented with a touch screen control that is activated when touched by a user. Alternatively, in any of the embodiments, one or more of the controls that are carried out via a touch screen can be replaced or supplemented with a dedicated control 82 that carries out the same function when activated by a user.

Through either dedicated controls 82 and/or a touch screen display (e.g. display 88), control panel 76 enables a user to turn thermal control unit 22 on and off, select a mode of operation, select a target temperature for the fluid delivered to thermal wraps 24, select a patient target temperature, customize a variety of treatment, display, alarm, and other functions, and control still other aspects of thermal control unit 22, as is discussed in greater detail below. In some embodiments, control panel 76 may include a pause/event control, a medication control, and/or an automatic temperature adjustment control that operates in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 82.

In those embodiments where control panel 76 allows a user to select from different modes for controlling the patient's temperature, the different modes include, but are not limited to, a manual mode, a monitoring mode, an automatic mode, and/or a preset mode. The manual mode, automatic mode, and preset mode are all used for cooling and heating the patient. In the manual mode, a user selects a target temperature for the fluid that circulates within thermal control unit 22 and that is delivered to thermal wraps 24. Thermal control unit 22 then makes adjustments to heat exchanger 40 in order to ensure that the temperature of the fluid exiting supply hoses 26a is at the user-selected temperature. In the monitoring mode, thermal control unit 22 merely reports the temperature of the patient, but does not attempt to make adjustments to the patient's temperature. In the preset mode, thermal control unit 22 automatically follows a preset sequence of phases, wherein the patient's temperature is either adjusted or maintained at each of the phases.

When the user selects the automatic mode, the user selects a target patient temperature, rather than a target fluid temperature. After selecting the target patient temperature, controller 60 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, thermal control unit 22 utilizes a sensor module 74 that includes one or more patient temperature sensor ports 84 (FIGS. 2 & 3) that are adapted to receive one or more conventional patient temperature sensors or probes 86. The patient temperature sensors 86 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant or otherwise marketed as 400 series probes. In other embodiments, different types of sensors may be used with thermal control unit 22. Regardless of the specific type of patient temperature sensor used in thermal control system 20, each temperature sensor 86 is connected to a patient temperature sensor port 84 positioned on thermal control unit 22. Patient temperature sensor ports 84 are in electrical communication with controller 60 and provide current temperature readings of the patient's temperature.

Controller 60, in some embodiments, controls the temperature of the circulating fluid using closed-loop feedback from temperature sensor 56 (and, when operating in the automatic mode, also from patient temperature sensor(s) 86). That is, controller 60 determines (or receives) a target temperature of the fluid, compares it to the measured temperature from sensor 56, and issues a command to heat exchanger 40 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 60 multiplies the fluid temperature error by a proportional constant, determines the derivative of the fluid temperature error over time and multiplies it by a derivative constant, and determines the integral of the fluid temperature error over time and multiplies it by an integral constant. The results of each product are summed together and converted to a heating/cooling command that is fed to heat exchanger 40 and tells heat exchanger 40 whether to heat and/or cool the circulating fluid and how much heating/cooling power to use.

When thermal control unit 22 is operating in the automatic mode and/or preset modes, controller 60 may use a second closed-loop control loop that determines the difference between a patient target temperature and a measured patient temperature. The patient target temperature is input by a user of thermal control unit 22 using control panel 76. The measured patient temperature comes from a patient temperature sensor 86 coupled to one of patient temperature sensor ports 84 (FIG. 3). Controller 60 determines the difference between the patient target temperature and the measured patient temperature and, in some embodiments, uses the resulting patient temperature error value as an input into a conventional PID control loop. As part of the PID loop, controller 60 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 60 may utilize one or more PI loops, PD loops, and/or other types of control equations. In some embodiments, the coefficients used with the control loops may be varied by controller 60 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 60 implements the loop(s) multiple times a second in at least one embodiment, although it will be understood that this rate may be varied widely. After controller 60 has output a heat/cool command to heat exchanger 40, controller 60 takes another patient temperature reading (from sensor 86) and/or another fluid temperature reading (from sensor 56) and re-performs the loop(s). The specific loop(s) used, as noted previously, depends upon whether thermal control unit 22 is operating in the manual mode or automatic mode.

It will also be understood by those skilled in the art that the output of any control loop used by thermal control unit 22 may be limited such that the temperature of the fluid delivered to thermal wraps 24 never strays outside of a predefined maximum and a predefined minimum. Examples of such a predefined maximum temperature and predefined minimum temperature are disclosed and discussed in greater detail in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. The predefined minimum temperature is designed as a safety temperature and may be set to about four degrees Celsius, although other temperatures may be selected. The predefined maximum temperature is also implemented as a safety measure and may be set to about forty degrees Celsius, although other values may be selected.

In some embodiments of thermal control unit 22, such as the embodiment shown in FIG. 3, thermal control unit 22 also includes a reservoir valve 96 that is adapted to selectively move fluid reservoir 32 into and out of line with circulation channel 36. Reservoir valve 96 is positioned in circulation channel 36 between air remover 70 and valve 72, although it will be understood that reservoir valve 96 may be moved to different locations within circulation channel 36. Reservoir valve 96 is coupled to circulation channel 36 as well as a reservoir channel 98. When reservoir valve 96 is open, fluid from air remover 70 flows along circulation channel 36 to pump 34 without passing through reservoir 32 and without any fluid flowing along reservoir channel 98. When reservoir valve 96 is closed, fluid coming from air remover 70 flows along reservoir channel 98, which feeds the fluid into reservoir 32. Fluid inside of reservoir 32 then flows back into circulation channel 36 via valve 72. Once back in circulation channel 36, the fluid flows to pump 34 and is pumped to the rest of circulation channel 36 and thermal wraps 24 and/or bypass line 64. In some embodiments, reservoir valve 96 is either fully open or fully closed, while in other embodiments, reservoir valve 96 may be partially open or partially closed. In either case, reservoir valve 96 is under the control of controller 60.

In those embodiments of thermal control unit 22 that include a reservoir valve, thermal control unit 22 may also include a reservoir temperature sensor 100. Reservoir temperature sensor 100 reports its temperature readings to controller 60. When reservoir valve 96 is open, the fluid inside of reservoir 32 stays inside of reservoir 32 (after the initial drainage of the amount of fluid needed to fill circulation channel 36 and thermal wraps 24). This residual fluid is substantially not affected by the temperature changes made to the fluid within circulation channel 36 as long as reservoir valve 96 remains open. This is because the residual fluid that remains inside of reservoir 32 after circulation channel 36 and thermal wraps 24 have been filled does not pass through heat exchanger 40 and remains substantially thermally isolated from the circulating fluid. Two results flow from this: first, heat exchanger 40 does not need to expend energy on changing the temperature of the residual fluid in reservoir 32, and second, the temperature of the circulating fluid in circulation channel 36 will deviate from the temperature of the residual fluid as the circulating fluid circulates through heat exchanger 40.

In some embodiments, controller 60 utilizes a temperature control algorithm to control reservoir valve 96 that, in some embodiments, is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 60 utilizes a different control algorithm. In still other embodiments, thermal control unit 22 is modified to omit reservoir valve 96, reservoir channel 98, and reservoir temperature sensor 100. Thermal control unit 22 may also be modified such that reservoir 32 is always in the path of circulation channel 36. Still other modifications are possible.

It will be understood that the particular order of the components along circulation channel 36 of thermal control unit 22 may be varied from what is shown in FIG. 3. For example, although FIG. 3 depicts pump 34 as being upstream of heat exchanger 40 and air separator 70 as being upstream of pump 34, this order may be changed. Air separator 70, pump 34, heat exchanger 40 and reservoir 32 may be positioned at any suitable location along circulation channel 36. Indeed, in some embodiments, reservoir 32 is moved so as to be in line with and part of circulation channel 36, rather than external to circulation channel 36 as shown in FIG. 3, thereby forcing the circulating fluid to flow through reservoir 32 rather than around reservoir 32.

It will also be understood that the contents of the components of thermal control unit 22 may be changed from what is shown in FIG. 3, and that many embodiments of thermal control unit 22 may be implemented in accordance with the present disclosure that omit one or more of these illustrated components and/or that include additional or different components. Further details regarding the construction and operation of one embodiment of thermal control unit 22 may be found in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

In some embodiments, thermal wraps 24 are constructed in accordance with any of the thermal pads disclosed in any of the following commonly assigned U.S. patent applications: Ser. No. 15/675,061 filed Aug. 11, 2017, by inventors James Galer et al. and entitled THERMAL THERAPY DEVICES; Ser. No. 62/778,034 filed Dec. 11, 2018, by inventors Andrew M. Bentz et al. and entitled THERMAL SYSTEM WITH THERMAL PAD FILTERS; and Ser. No. 15/675,066 filed Aug. 11, 2017, by inventor James K. Galer and entitled THERMAL SYSTEM, the complete disclosures of all of which are incorporated herein by reference. Still other types of thermal wraps 24 may be used with thermal control system 20, and thermal control unit 22 may be modified from its construction described herein in order to accommodate the particular thermal therapy pad(s) it is used with.

Memory 80 (FIG. 3) may be any type of conventional non-volatile memory, such as, but not limited to flash memory, one or more hard drives, one or more EEPROMs, etc. Memory 80 may also be implemented to include more than one of these types of memories in combination. In the embodiment shown in FIG. 3, memory 80 of thermal control unit 22 includes a plurality of items stored therein, such as one or more sets of each of the following: alarm conditions 102, alarm characteristics 172, therapy presets 106, user data 108, location data 110, and graphing data 112. These items are able to be entered into memory 80 locally via control panel 76 and/or are transmitted to thermal control unit via network transceiver 90. When sent via network transceiver, these items may originate from a thermal management server 150, a patient support apparatus server 160, a user device 170, and/or another device. Memory 80 may also include additional information beyond that shown in FIG. 3, such as, but not limited to, one or more algorithms for carrying out its functions, data recorded during the operation of thermal control unit 22, and/or other data. Memory 80 may also, in some embodiments, omit any one or more of the specific data items shown in FIG. 3.

Off-board transceiver 90 is adapted to communicate with one or more off-board devices, such as, but not limited to, a wireless access point of a local area network 122, a network cable of a local area network, and/or other devices. In the embodiment shown in FIG. 3, transceiver 90 is a Wi-Fi radio communication module configured to wirelessly communicate with one or more wireless access points 118 of a local area network 122. In such embodiments, transceiver 90 may operate in accordance with any of the various IEEE 802.11 standards (e.g. 802.11b, 802.11n, 802.11 g, 802.11ac, 802.11ah, etc.). In other embodiments, transceiver 90 may include, either additionally or in lieu of the Wi-Fi radio and communication module, a wired port for connecting a network wire to thermal control unit 22. In some such embodiments, the wired port accepts a category 5e cable (Cat-5e), a category 6 or 6a (Cat-6 or Cat-6a), a category 7 (Cat-7) cable, or some similar network cable, and transceiver 90 is an Ethernet transceiver. In still other embodiments, transceiver 90 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with transceiver 90, controller 60 is able to communicate with the local area network 122 (FIG. 3) of a healthcare facility in which the thermal control unit 22 is positioned. When transceiver 90 is a wireless transceiver, it communicates with local area network 122 via one or more wireless access points 118. When transceiver 90 is a wired transceiver, it communicates directly via a cable coupled between thermal control unit 22 and a network outlet positioned within the room of the healthcare facility in which thermal control unit 22 is positioned.

Local area network 122 typically includes a plurality of servers, the contents of which will vary from healthcare facility to healthcare facility. In general, however, most healthcare facilities will include, among other servers, an electronic medical records (EMR) server 124, which may be a conventional server. In addition to EMR server 124, local area network 122 includes a patient support apparatus server 160 that is in communication with one or more patient support apparatuses 116 (FIGS. 1 and 3). Network 122 is also in communication with a temperature management server 150. In the example shown in FIG. 3, network 122 is in communication with the Internet 152 and temperature management server 150 is coupled to network 122 via the Internet 152. In other arrangements, temperature management server 150 could be located locally (e.g. within a healthcare facility's premises) such that it was hosted directly on the healthcare facility's network 122. Still further, in some embodiments, temperature management server 150 and patient support apparatus server 160 may be combined (either locally or remotely).

In addition to the aforementioned servers 124 and 160, network 122 may include one or more additional servers and/or other network appliances. For example, as mentioned, network 122 may include an Internet server and/or an Internet gateway that couples network 122 to the Internet 152, thereby enabling a remotely position temperature management server 150 to communicate with network 122, thermal control units 22, patient support apparatus server 160, EMR server 124, and/or other servers on network 122. In some embodiments, EMR server 124 may be located remotely from the premises in which patient support apparatuses 116 and thermal control units 22 are located. In such embodiments, EMR server 124 is coupled to the Internet 152 and temperature management server 150 communicates with EMR server 124 over the Internet 152.

Another type of server that may be included with computer network 122 is a location server (not shown) that is adapted to monitor and record the current locations of thermal control units 22, patients, patient support apparatuses 116, and/or caregivers within the healthcare facility. Such a location server may communicate with the thermal control units 22 via access points 118 and transceivers 90. Network 122 may also include a conventional Admission, Discharge, and Tracking (ADT) server that allows thermal control units 22 and/or temperature management server 150 to retrieve information identifying the patient undergoing thermal therapy. Still further, healthcare network 122 may further include one or more conventional work flow servers and/or charting servers that assign, monitor, and/or schedule patient-related tasks to particular caregivers, and/or one or more conventional communication servers that forward communications to particular individuals within the healthcare facility, such as via one or more user devices 170 (smart phones, tablet computers, pagers, beepers, laptops, etc.). The forwarded communications may include data and/or alerts that originate from thermal control units 22 and/or elsewhere.

In some embodiments, local area network 122 may include any one or more of the servers described and disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which is incorporated herein by reference. Further, in in such embodiments, thermal control units 22 may be configured to communicate with the servers on network 122 in any of the manners disclosed in the '587 PCT application, and/or to retrieve and/or share any of the information disclosed in the '587 PCT application.

As noted, temperature management server 150 and patient support apparatus server 160 are adapted to communicate with one or more user devices 170 (FIG. 3). User devices 170 include any computing devices that are adapted to communicate with network 122, such as, but not limited to, smart phones, tablet computers, laptop computers, desktop computers, smart televisions, computer displays, and/or other devices. As will be discussed in greater detail below, temperature management server 150 is adapted to communicate with user devices 170 to provide information to the users of those devices regarding thermal therapies carried out using one or more thermal control units 22. In addition, temperature management server 150 may be configured to allow user devices 170 to send remote commands to thermal control units 22, remotely update the software of thermal control units 22, remotely install new thermal presets 106, and/or perform other actions. In some embodiments, temperature management server 150 also communicates with patient support apparatus server 160 such that it is able to provide both thermal control data to user devices 170 and patient support apparatus data to user devices 170.

Although not shown in FIG. 3, thermal control unit 22 includes a clock/calendar that communicates with controller 60. The clock/calendar not only measures the passage of time, but it also keeps track of the calendar day (and year). Controller 60 may use the outputs from clock/calendar day to time stamp the data it gathers and sends to temperature management server 150, as well as for other purposes (e.g. implementing one or more therapy presets 106). The clock/calendar may be any conventional timing device that is able to keep track of the passage of time, including the calendar day and year.

In the embodiment shown in FIG. 3, thermal control unit 22 further includes a location sensor 92. Location sensor 92 automatically detects the location of thermal control unit 22 within a healthcare facility. Location sensor 92 may take on a variety of different forms. For example, in one embodiment, thermal control unit 22 includes a WiFi transceiver (which may be the same as transceiver 90 or may be an additional/separate transceiver) that communicates with the healthcare facility's local area network via the network's wireless access points 118, and controller 60 determines its location relative to the known locations of these access points based upon the detected signal strengths from these access points. In another example, location sensor 92 and controller 60 may determine their location using any of the same methods and/or sensors for determining patient support apparatus location that are disclosed in commonly assigned U.S. Pat. No. 9,838,836 issued Dec. 5, 2017, to inventors Michael J. Hayes et al. and entitled PATIENT SUPPORT APPARATUS COMMUNICATION SYSTEMS, the complete disclosure of which is incorporated herein by reference. Still other automatic location detection methods may be used, including, but not limited to, the use of cellular network trilateration and/or Global Positioning System (GPS) sensors.

In addition to the patient temperature sensor(s) 86, the water temperature sensor 56, the reservoir temperature sensor 100 (if included), and the location sensor 92 (if included), thermal control unit 22 may include still more sensors that are positioned within main body 30, and/or that are positioned outside of main body 30 and in communication with main controller 60 (FIG. 3). Such off-board sensors (e.g. outside of main body 30) may communicate with main controller 60 via one or more of the auxiliary sensor ports 94 and/or via one or more of the transceivers 90. Each auxiliary sensor port 94 is adapted to receive outputs from an off-board auxiliary sensor 128. The auxiliary sensors 128, as well as any additional sensors onboard thermal control unit 22, provide additional data to controller 60 regarding the patient during a thermal therapy session. Controller 60 is configured to utilize the additional data either for use in one or more algorithms that are currently being used by thermal control unit 22 to control the patient's temperature, or for potential future use in one or more improved algorithms that are determined, after analysis, to provide improved results for the thermal therapy sessions carried out using thermal control unit 22. The additional data, when not currently used for controlling thermal control unit 22, may be analyzed by temperature management server 150 in order to determine if the additional data can improve the performance of thermal control unit 22, and/or if it can predict the occurrence of one more undesired events, such as, for example, patient shivering or overshooting the target temperature of the patient.

Auxiliary ports 94 (FIGS. 2 & 3) may take on a variety of different forms. In one embodiment, all of the ports 94 (if there are more than one) are of the same type. In another embodiment, thermal control unit 22 includes multiple types of ports. In any of these embodiments, the ports 94 may include, but are not limited to, a Universal Serial Bus (USB) port, an Ethernet port (e.g. an 8P8C modular connector port, or the like), a parallel port, a different (from USB) type of serial port, etc. Ports 94 may also or alternatively be implemented wirelessly, such as via a WiFi transceiver, a Bluetooth transceiver, a ZigBee transceiver, etc. In these latter embodiments, one or more of transceivers 90 may be incorporated into sensor module 74 and in communication with one or more of the ports 94.

Thermal control unit 22 may be configured to accept a number of different types of auxiliary sensors 128 via input ports 94. Such sensors include, but are not limited to, the following: an end tidal carbon dioxide (ETCO₂) sensor that detects ETCO₂ levels of the patient; a respiration rate sensor that senses the respiration rate of the patient; a blood pressure sensor that detects the blood pressure of the patient; a heart rate sensor that detects the heart rate of the patient; a scale sensor that detects the patient's weight and/or movement; an electrolyte sensor that detects levels of one or more electrolytes (e.g. potassium) in the patient's blood; a pulse wave velocity sensor that detects the patient's pulse wave velocity; an oxygen saturation level (SpO₂) sensor that detect oxygen saturation levels of the patient; a bioimpedance sensor that detects a bioimpedance of the patient, such as, but not limited to, the bioimpedance at one or more locations on the patient's body in contact with a thermal wrap 24; an electrocardiograms or that detects an electrocardiogram of the patient; a temperature change sensor that detects a rate of temperature change of the patient; one or more sensors that are integrated into one or more of the thermal wraps 24 and that detect characteristics of the thermal wraps 24 and/or of the patient (e.g. temperature sensors built into the thermal wraps 24); one or more temperature sensors that detect one or more peripheral temperatures of the patient (as opposed to the core temperature sensed by sensor 86); an ultrasonic sensor adapted to detect attenuation levels of ultrasonic waves traveling through at least a portion of the patient's body; a near infrared sensor adapted to detect attenuation levels of near infrared waves traveling through at least a portion of the patient's body; a perfusion sensor adapted to detect a patient's blood perfusion levels; a vibration sensor (e.g. accelerometer) adapted to detect vibrations of the patient, such as due to shivering; a thermal image sensor adapted to capture thermal images of the patient; an electromyograph adapted to detect electrical activity in the patient's muscles; one or more air quality sensors (e.g. air pressure, humidity, air temperature, air volume, etc.) that measure characteristics of the air breathed by the patient and/or the ambient air; and/or still other sensors.

When thermal control unit 22 is utilized with a respiration rate sensor and/or a heart rate sensor coupled to one or more auxiliary ports 94, these sensors may be directly attached to the patient and/or they may be adapted to passively monitor these parameters without direct attachment to the patient. In some embodiments, passive heart rate sensors and/or respiration rate sensors may be built directly into patient support apparatus 116 that communicate their outputs to thermal control unit 22. One example of such sensor are disclosed in commonly assigned U.S. Pat. No. 7,699,784 filed Jul. 5, 2007, by inventors David Wan Fong et al. and entitled SYSTEM FOR DETECTING AND MONITORING VITAL SIGNS, the complete disclosure of which is incorporated herein by reference. Still other types of both passive and non-passive vital sign sensors may be used.

When thermal control unit 22 is utilized with any one or more of an end tidal carbon dioxide (ETCO₂) sensor, a blood pressure sensor, an oxygen saturation level sensor, a respiration rate sensor, a heart rate sensor, an electrolyte sensor, a pulse wave velocity sensor, a bioimpedance sensor, an electrocardiogramsor, or a rate of temperature change sensor coupled to one or more auxiliary ports 94, such sensors may be of the same type, and/or utilized in the same or similar manners, as those disclosed in more detail in commonly assigned U.S. patent application Ser. No. 16/912,244 filed Jun. 25, 2020, by inventors Gregory S. Taylor et al. and entitled THERMAL SYSTEM WITH USER INTERFACE CUSTOMIZATION, the complete disclosure of which is incorporated herein by reference. Still other types of these sensors may be used.

When thermal control unit 22 is utilized with any one or more sensors that are integrated into one or more of the thermal wraps 24 and that are coupled to one or more auxiliary ports 94, such sensors may be of the same type, and/or utilized in the same or similar manners, as those disclosed in more detail in commonly assigned U.S. patent application Ser. No. 15/675,066 filed Aug. 11, 2017, by inventor James Galer and entitled THERMAL SYSTEM, the complete disclosure of which is incorporated herein by reference. Still other types of these sensors may be used.

When thermal control unit 22 is utilized with any one or more of an ultrasonic sensor, an infrared sensor, a perfusion sensor, and/or a peripheral patient temperature sensor coupled to one or more auxiliary ports 94, such sensors may be of the same type, and/or utilized in the same or similar manners, as those disclosed in more detail in commonly assigned PCT patent application PCT/US2018/066114 filed Dec. 18, 2018, by Applicant Stryker Corporation and entitled THERMAL SYSTEM WITH PATIENT SENSOR(S), the complete disclosure of which is incorporated herein by reference. Still other types of these sensors may be used.

When thermal control unit 22 is utilized with any one or more of a vibration sensor, a thermal image sensor, and/or an electromyograph coupled to one or more auxiliary ports 94, such sensors may be of the same type, and/or utilized in the same or similar manners, as those disclosed in more detail in commonly assigned U.S. patent application Ser. No. 15/820,558 filed Nov. 22, 2017, by inventors Gregory S. Taylor et al. and entitled THERMAL SYSTEM, the complete disclosure of which is incorporated herein by reference. Still other types of these sensors may be used.

When thermal control unit 22 is utilized with any one or more air quality sensors (air pressure, humidity, air temperature, air volume, etc.) coupled to one or more auxiliary ports 94, such sensors may be of the same type, and/or utilized in the same or similar manners, as those disclosed in more detail in commonly assigned PCT patent application PCT/US2018/064685 filed Dec. 10, 2018, by Applicant Stryker Corporation and entitled THERMAL CONTROL SYSTEM, the complete disclosure of which is incorporated herein by reference. Still other types of these sensors may be used.

When thermal control unit 22 is utilized with a scale sensor coupled to one or more auxiliary ports 94, the scale sensor may be built into patient support apparatus 116 and/or separate from patient support apparatus 116. In some embodiments where the scale sensor is built into patient support apparatus 116, the scale sensor may include any of the load cells and/or other movement sensors disclosed in commonly assigned U.S. patent application Ser. No. 14/873,734 filed Oct. 2, 2015, by inventors Marko Kostic et al. and entitled PERSON SUPPORT APPARATUSES WITH MOTION MONITORING, and/or in commonly assigned U.S. patent application Ser. No. 15/346,779 filed Nov. 9, 2016, by inventors Marko Kostic et al. and entitled PERSON SUPPORT APPARATUSES WITH ACCELERATION DETECTION, the complete disclosures of both of which are incorporated herein by reference.

It will be understood that the sensors incorporated into thermal control unit 22 may be augmented and/or otherwise modified from what is shown in FIG. 3. For example, thermal control unit 22 may include one or more of the following sensors positioned inside main body 30: one or more input fluid temperature sensors that measure the temperature of the fluid returning to inlet manifold 78 (e.g. a single inlet temperature sensor or multiple inlet temperature sensors that measure the fluid temperature for each individual inlet port 62); one or more water quality sensors that measure the cleanliness and/or other characteristics of the fluid (e.g. water) that is circulating in circulation channel 36 and being delivered to the thermal wraps 24; one or more flow rate sensors that measure the flow rate of fluid through circulation channel 36 (and/or that measure individual flow rates through each of the outlet ports 58 and/or through each of the inlet ports 62); one or more valve sensors that detect the position of one or more valves within thermal control unit 22; and/or still other types of sensors.

In some embodiments, thermal control unit 22 and/or temperature management server 150 are configured to receive additional information from EMR server 124, user devices 170, and/or other sources. Such additional information may include, but is not limited to, any one or more of the following: the patient's age, weight, height, BMI, BSA, and/or other patient information; medication information indicating what medications patient 28 is on or has received prior to, or during, the thermal therapy session; location information that indicates the current location of thermal control unit 22; caregiver identification information that identifies which caregiver is currently using thermal control unit 22; treatment information identifying the diagnosis of patient 28 and/or the intended use for the thermal therapy session (e.g. for neurotrauma, cardiac arrest, etc.); and/or still other types of information.

FIG. 4 shows one manner in which the layout of control panel 76 of thermal control unit 22 can be implemented, including an illustrative arrangement of dedicated controls 82 that are positioned around display 88. Controls 82 include a therapy pause control 82a that, when pressed, pauses the therapy being performed by thermal control unit 22. To resume therapy, a user presses and holds down on the therapy pause control 82. A selection control 82b allows a user to switch between displaying the temperatures in Fahrenheit and Celsius by pressing on control 82b, which acts as a toggle switch between the two different units of measurement. A power control 82c will turn on and off thermal control unit 22 when pressed. When a user first presses a lock control 82d, the screen will be locked and pressing on any areas of the screen will not change any settings, or otherwise cause thermal control unit 22 to react to the pressing. In order to unlock the touchscreen, a user presses down and holds the lock control 82d for at least two seconds. An audio pause control 82e, when pressed, silences any audible alarms for a predetermined period of time, such as ten minutes. Any alarms will still result in a visual display of the alarm on display 88, but will not result in any audible indications while the audio pause is in effect. Any alarms will also still be sent via transceiver 90 to temperature management server 150, regardless of the status of audio pause control 82e. Control panel 76 further includes four therapy mode controls 82f, 82g, 82h, and 82i. Pushing down on control 82f will cause thermal control unit 22 to act in the automatic mode (described previously). Pushing down on control 82g will cause thermal control unit 22 to act in the manual mode (also described previously). Pushing down on control 82h will cause thermal control unit 22 to act in a monitor mode (not described previously). In the monitor mode, thermal control unit 22 does not circulate fluid or regulate the fluid's temperature, but instead merely monitors the temperature(s) input into thermal control unit 22 via the patient temperature probe ports 84 and issues any alarms if the temperatures change beyond any user-defined thresholds.

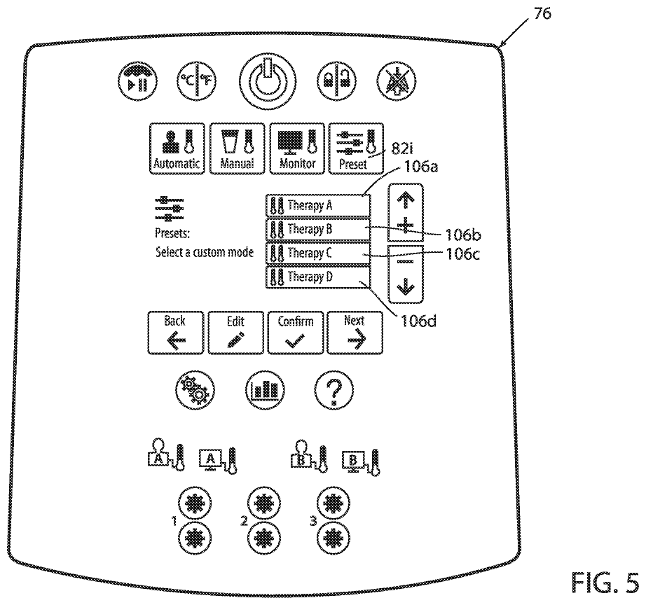

Pushing down on control 82i causes thermal control unit 22 to display a plurality of therapy presets 106, several examples of which are shown in FIG. 5. Therapy presets 106 refer to predefined sequences of patient temperature control that are stored in memory 80 of thermal control unit 22. Each preset includes a plurality of temperature phases that are sequentially followed by thermal control unit 22 and that can be defined by a user of thermal control system 20. As will be discussed in greater detail below, thermal control system 20 allows authorized personnel to define thermal preset 106 using their user devices 170. These new presets 106 may then be sent by temperature management server 150 to thermal control unit 22 where they will then be displayed in response to the user pressing on control 82i. Authorized users may customize the duration of each phase in the preset 106, the rate at which the patient is warmed or cooled during each phase, and/or the alarms and/or notifications that occur at each phase.

A back control 82j (FIG. 4) causes controller 60 to change what is displayed on display 88 to that which was displayed thereon immediately prior to the pressing of the back control 82j. An edit control 82k enables the user to edit current settings when pressed, or exit or cancel, depending upon the context of the information displayed on LCD display 88. A confirm control 821, when pressed, allows a user to confirm a selection made by the user of information displayed on display 88. A forward control 82m shifts, when pressed, what is displayed on display 88 to the next sequential screen.

Control 82n is a settings icon that, when pressed, displays a summary of the current settings of thermal control unit 22, and may also display controls for changing one or more of the settings. Pressing on control 820 will graphically display one or more user-selected parameters on display 88, such as, but not limited to, the measured and recorded patient temperatures, the target temperature, the fluid temperature and working capacity.

Although not illustrated in FIG. 4, control panel 76 may further includes several additional controls and/or indicia. For example, in some embodiments, control panel 76 may include port icons that correspond to outlet ports 58 and that indicate, based on their illumination state (color, on/off, etc.), whether each respective port 58 is active, inactive, blocked, and/or in another state. Further details regarding one manner in which such port icons may be displayed, as well as the information conveyed by such icons, may be found in commonly assigned U.S. Pat. No. 10,390,992 issued to Hopper et al. on Aug. 27, 2019, and entitled THERMAL CONTROL SYSTEM, the complete disclosure of which is incorporated herein by reference. Thermal control unit 22 may also include any features and/or functions of the thermal control units disclosed in the aforementioned '992 patent.

Among other functions, controls 82 and/or touchscreen display 88 of control panel 76 allow a user to perform one or more of the following functions: activate/deactivate one or more of a plurality of alarms; choose the characteristics of each of the available alarms; select a therapy preset 106 for implementing the thermal therapy; define the characteristics of the selected thermal therapy; instruct thermal control unit 22 to display graph information about a thermal therapy session; select what information is included within the graph information; define characteristics of the graph information; control what information is received from any off-board sensors that are adapted to communicate with one of the transceivers 90; control what information is recorded, displayed, and/or transferred to other devices during a thermal therapy session; communicate with EMR server 124 and remote computing device 126; receive information about a patient undergoing thermal therapy; start, stop, and pause a thermal therapy session; analyze outputs from one or more sensors to determine if the patient is shivering; and other functions.

Display 88 of control panel 76 (FIG. 4) is configured to display a plurality of different screens thereon. As noted, display 88 may be a touchscreen-type display, although it will be understood that a non-touchscreen display may alternatively be used. Display 88 displays one or more visual indicators, one or more controls, and/or one or more control screens, and/or other types of information, as will be discussed more below. Display 88 may comprise an LED display, an OLED display, or another type of display. Display 88 may be configured to have its brightness level adjusted. That is, the amount of light emitted from display 88 can be varied by a controller included within thermal control unit 22. In some embodiments, the brightness is adjusted based on one or more ambient light sensors, such as is disclosed in commonly assigned U.S. patent application Ser. No. 63/31,973 filed May 29, 2020, by inventors Frank Lee et al. and entitled PATIENT SUPPORT APPARATUS WITH AUTOMATIC DISPLAY CONTROL, the complete disclosure of which is incorporated herein by reference. Still other types of brightness control, sensors, and/or sensing systems may be used.

FIG. 5 illustrates control panel 76 after the user has selected the preset control 82i. As shown therein, control panel 76 displays four presets 106a-d. The number of presets 106 may be less than the four shown in FIG. 5, or it may be greater than four. The number of preset 106 will correspond to the number of presets that authorized personnel have defined using their user devices 170 and sent to thermal control unit 22. As will be discussed in greater detail below, temperature management server 150 is configured to execute a software application that allows it to communicate with user devices 170 such that users of devices 170 are able to define thermal presets 106, send them to server 150, and server 150 is in turn able to send them to thermal control units 22. The number of presets 106 that appear on display 88 of control panel 76 will therefore vary according to the number of presets 106 that a particular healthcare facility has defined and forwarded to thermal control unit 22.

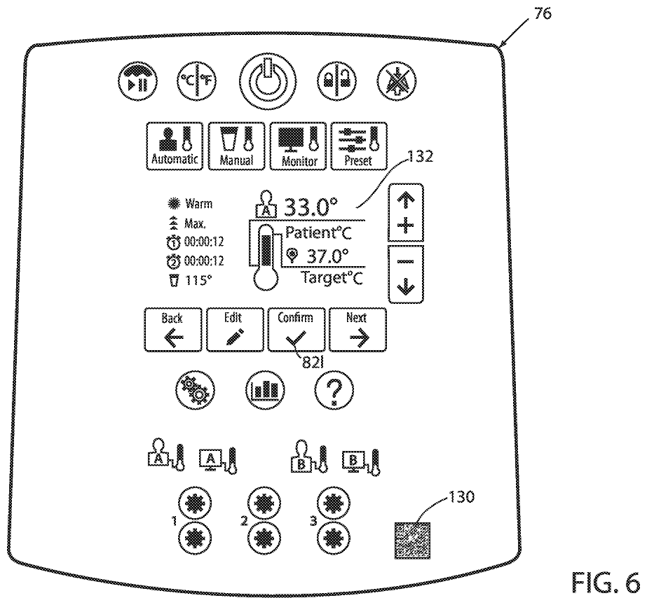

FIG. 6 illustrates control panel 76 after the user has selected a particular preset 106 and pressed the confirm button 821. Controller 60 of thermal control unit 22 is configured to start a thermal therapy session in response to the user pressing confirm button 821. In addition, controller 60 is configured to display a QR code 130 (or other type of visual code, such as, but not limited to, a bar code) on control panel 76 in response to the user pressing confirm button 82. The QR code 130 that controller 60 generates in response to the commencement of a thermal therapy session (which commences in response to confirm button 821 being pressed after a therapy mode has been selected) is a unique code that contains a unique session ID embedded in the code 130. In other words, controller 60 is configured to generate a unique therapy session ID for every thermal therapy session it performs, and that therapy ID is embedded in QR code 130. As will be discussed in more detail below, the display of QR code 130 on control panel 76 allows a caregiver to scan the QR codes 130 user a conventional user device 170 (e.g. a smart phone) that is able to decipher the QR code 130. The caregiver can then scan a patient's bracelet, wristband, or other user ID, using that same device 170, thereby enabling a particular patient ID to be associated with a particular thermal therapy session. The order of scanning the patient's ID and QR code 130 can, of course be reversed. Manual means for associating a particular therapy session ID with a particular patient may also, or alternatively, be used.

The particular therapy session ID that controller 60 generates for a particular thermal therapy session is forwarded by controller 60 to temperature management server 150 (via transceiver 90 and network 122 (and, in some cases, through the Internet 152). All of the data that thermal control unit 22 generates during that particular thermal therapy session is thus associated with a particular therapy session ID. Server 150 stores this data and therapy session ID and makes it available to users of devices 170. In some instances, server 150 also display information on user devices 170 about the patient associated with the particular thermal therapy session.

As shown in FIG. 6, once a thermal therapy session commences, controller 60 may be configured to display a therapy status indicator 132 on control panel 76 that displays information about the currently ongoing thermal therapy session. In the example of FIG. 6, indicator 132 identifies the patient's current temperature (33° C.), which is sensed by patient temperature probe 86 and fed to thermal control unit 22 (via port(s) 84), as well as the current target temperature for the patient (37° C.). Other information may also, or alternatively, be included within therapy status indicator 132.

As was noted, patient temperature server 150 is adapted to execute one or more software applications that communicate with thermal control units 22 and one or more user devices 170. In some embodiments, the software application(s) also enable server 150 to communicate with patient support apparatus server 160, EMR server 124, and/or other servers or devices. For purposes of the following description, server 150 and the software application(s) it executes will be referred to interchangeably. That is, references to server 150 performing a particular action will be understood to mean server 150 is programmed according to one or more software applications to perform that particular action.

In some embodiments, server 150 is configured to implement a web browser-based communication system between user devices 170 and itself. In such embodiments, user devices 170 include a conventional web browser that is used to access server 150. When so accessed, user devices 170 are able to see data from, edit data from, issue commands to, and/or otherwise interact with server 150 and/or thermal control units 22. When server 150 implements such a web browser-based communication system, it is not necessary for user devices 170 to include any specialize software applications to access temperature management server 150 (other than a conventional web browser, which is typically included as a standard software application on most user devices 170).

In other embodiments, server 150 is configured to communicate with a native software application that is executed on user devices 170. That native software application is specially designed for communication with server 150. In these embodiments, the user must first download the native software application onto his or her user device 170 before they are able to communicate with server 150. In some embodiments, server 150 is configured to implement both a web browser-based communication system and a native software application communication system, thereby enabling users within a given healthcare facility to access thermal control system 20 using either a conventional web browser or a native software application.

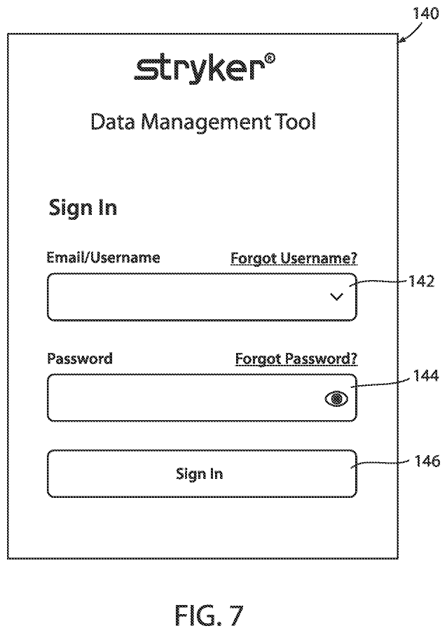

FIG. 7 illustrates one example of a login screen 140 that server 150 causes to be displayed (either through the web browser-based communications or through the native app, as mentioned above) on a display of a user device 170 when the user wishes to access thermal control system 20. Login screen 140 includes a user ID field 142 and a password field 144 into which the user types his/her username and password, respectively. After entering this information, the user selects the sign-in control 146. In response to the user selecting the sign-in control 146, server 150 checks the user's credentials to see if they are an authorized user. In addition, server 150 checks to see what role that particular user is assigned. The different roles that a particular user may be assigned to may vary in different embodiments. In one embodiment, each user is assigned one of three roles: a nurse, a nurse manager, or an administrator. Server 150 is configured to display different information and/or provide different functionality to each of the different roles.

In some embodiments, server 150 communicates with EMR server 124 to determine a particular user's role. In such embodiments, server 150 requests from EMR server 124 the job title, or other user class, that the healthcare facility has assigned to a particular user. Server 150 is configured in such embodiments to translate, if necessary, the healthcare facility's job title and/or user class into one of the particular roles that are defined for thermal control system 20. For example, a particular user who the healthcare facility classifies as a doctor may be translated by server 150 into the “administrator” or “nurse manager role.” Another user who the healthcare facility classifies as a nurse assistant or physician assistant might be translated into a nurse role by server 150, or some other role. When server 150 is initially installed, it is programmed to classify all of the job titles, or other classes, that the particular healthcare facility uses for its employees into corresponding thermal control system roles.

In some embodiments, server 150 is alternatively, or additionally, configured to determine a user's role without consulting EMR server 124. In such embodiments, server 150 may include its own table, or other data structure, that defines the roles of individual users. Alternatively, or additionally, server 150 may be configured to communicate with another server on network 122 that contains job titles or worker classes for individual users, and to then use those job titles or worker classes for translation into roles for thermal control system 20.

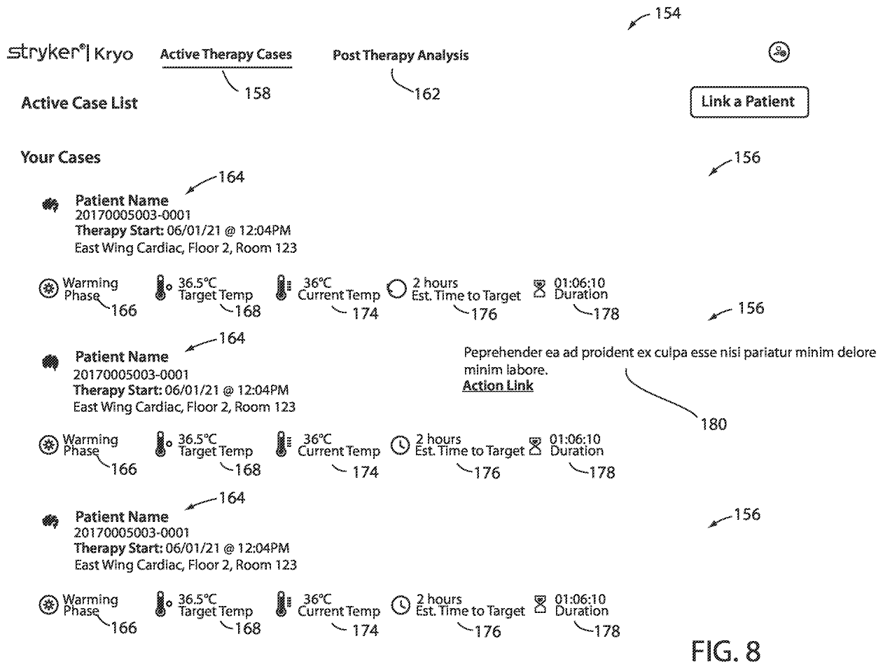

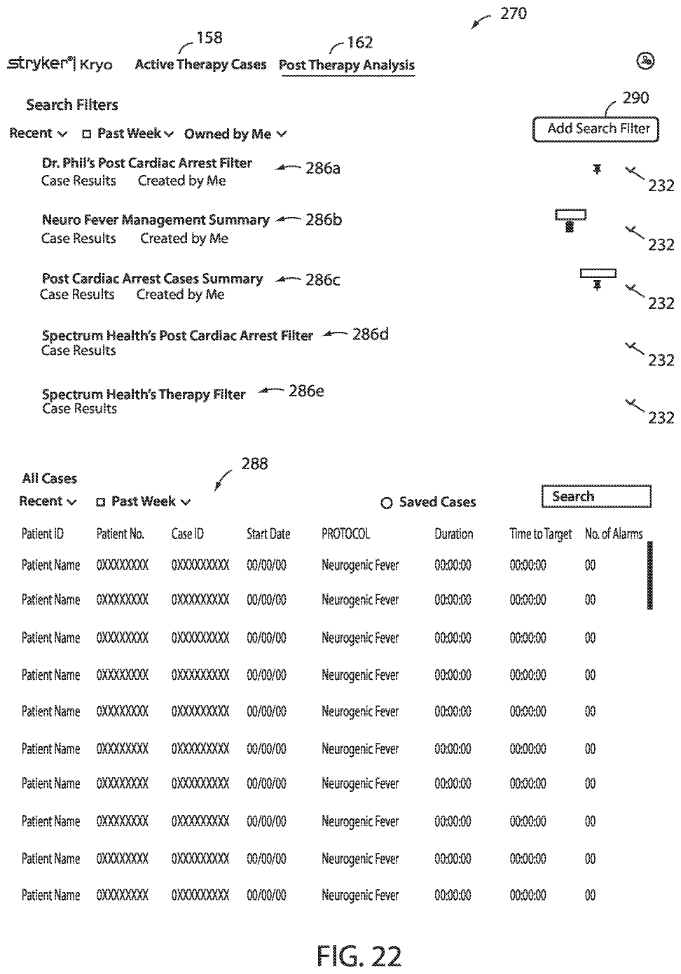

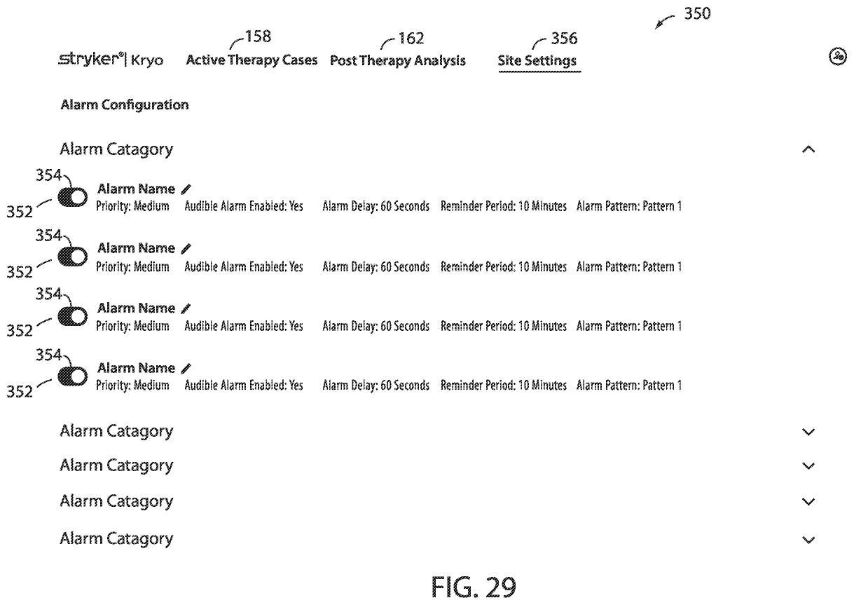

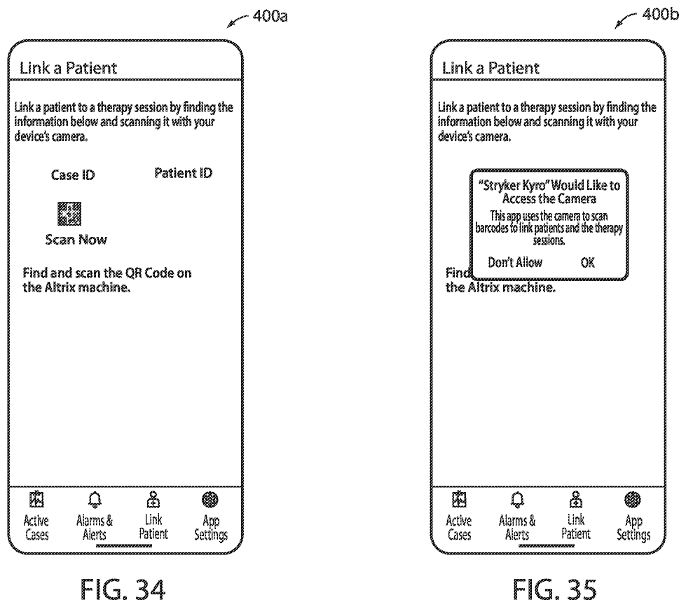

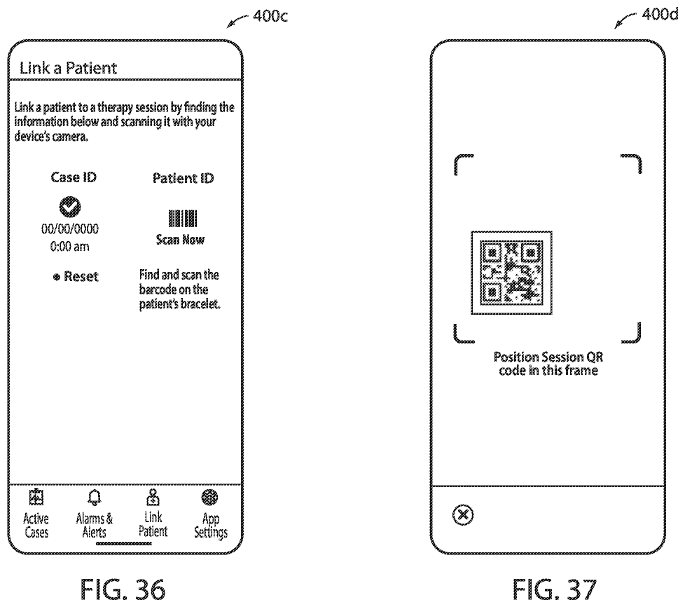

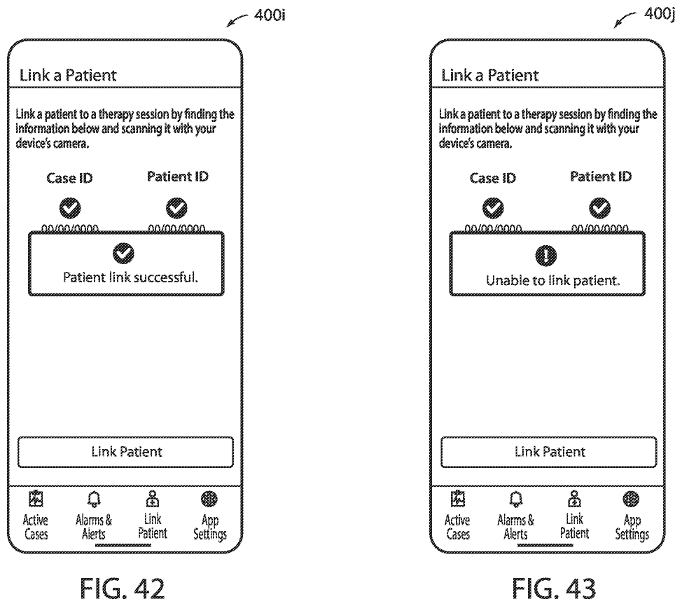

If server 150 determines that a particular user is an authorized user, based on the login credentials entered through login screen 140 (FIG. 7), server 150 is configured to grant that particular user access to various features of thermal control system 20. In general, thermal control system 20 provide four main classes of screens for viewing and/or entering data. These four main classes of screens are (1) active therapy screens, (2) post-therapy screens, (3) site setting screens, and (4) patient association screens. The active therapy screens are screens that display information about one or more thermal therapy sessions that are currently taking place using one or more thermal control units 22. The post-therapy screens are screens that display information from past thermal therapy sessions that were performed using one or more thermal control units 22. The site settings screens are screens that display administrative settings and/or other information that allows authorized personnel at a given healthcare facility (i.e. a specific site) to customize certain aspects of thermal control system 20 to their particular liking. The patient association screens are screens that may be used to associate a particular patient with a particular thermal therapy session. FIGS. 8-21 display examples of active therapy screens of thermal control system 20. FIGS. 22-28 display examples of post-therapy screens of thermal control system 20. FIGS. 29-33 display examples of site setting screens of thermal control system 20, and FIGS. 34-43 display examples of patient association screens of thermal control system 20. All of these screens (FIGS. 8-43) are displayed on the displays of one or more user devices 170 that are in communication with server 150.

FIG. 8 illustrates an example of an active case overview screen 154 that includes a plurality of case windows 156. Each case window 156 corresponds to a thermal therapy session that is currently being performed by a thermal control unit 22. Overview screen 154 also includes an active case indicator 158 and a post therapy indicator 162. Active case indicator 158, when active (e.g. underlined in the examples shown herein), indicates that the currently displayed screen refers to one or more thermal therapy sessions that are currently active. Post therapy indicator 162, when active (e.g. underlined in the examples shown in FIGS. 22-28), indicates that the currently displayed screen refer to past thermal therapy session.

Each case window 156 on case overview screen 154 (FIG. 8) provides high level details regarding the corresponding active thermal therapy session. These high-level details may vary in different embodiments. In the example shown in FIG. 8, each case window 156 includes a patient name indicator 164, a phase identifier 166, a patient target temperature 168, a current patient temperature 174, an estimate 176 of the amount of time remaining until the patient achieves his or her target temperature, and a duration indicator 178 that indicates how long that particular thermal therapy session has been ongoing. One or more of case windows 156 may also include a note window 180 if a user of system 20 has added one or more notes for that particular thermal therapy session. As will be discussed in greater detail below, a user of system 20 can add notes regarding the thermal therapy sessions that are performed using thermal control units 22. These notes are recorded in system 20 and, in some embodiments, transferred to EMR server 124.

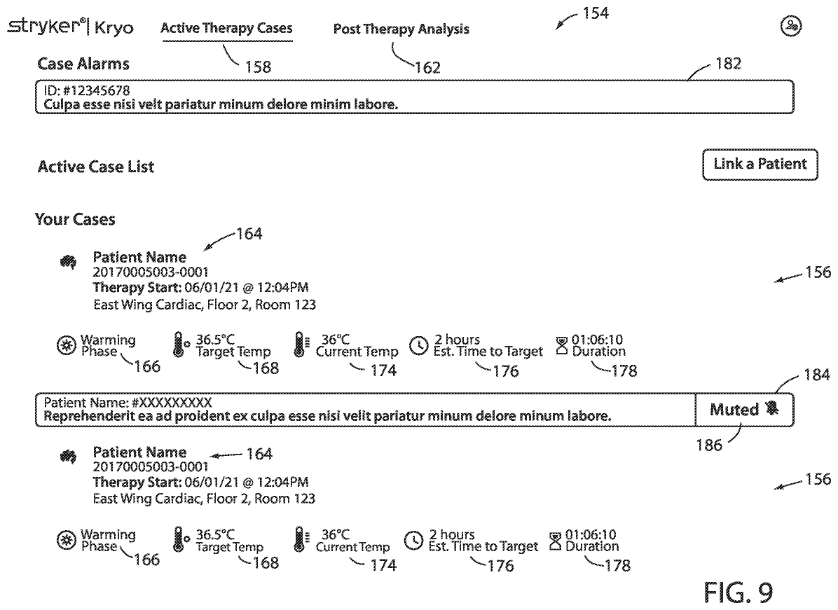

FIG. 9 illustrates active case overview screen 154 when one or more of the currently active thermal therapy sessions includes an alarm associated with it. In such cases, server 150 is configured to cause the user device to display an alarm window 182 to be displayed at the top of screen 154. Server 150 also adds an alarm header 184 to the top of the case window 156 that includes the alarm. Still further, server 150 may be configured to include an alarm indicator 186 that indicates whether the alarm is currently muted or not. In some embodiments, if the user pushes on alarm indicator 186, they can remotely mute or unmute the alarms on the corresponding thermal control unit 22. That is, in response to the user pressing alarm indicator 186, server 150 sends a command to the corresponding thermal control unit 22 instructing it to mute or unmute the associated alarm. Controller 60 implements the mute or unmute command.

Alarm window 182 and/or alarm header 184 (FIG. 9) may include additional information about the particular alarm associated with that particular window 182 or header 184. This additional information may include such things as the name of the alarm, when the alarm occurred, how long the alarm has lasted, etc.

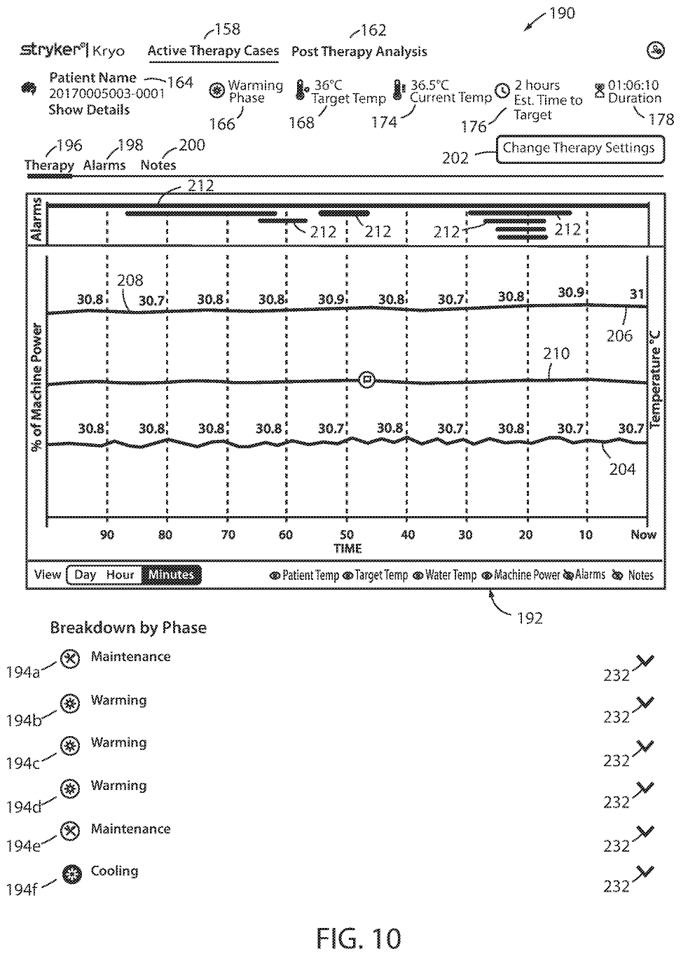

Server 20 is configured to display additional information on user devices 170 about a particular thermal therapy session in response to the user pressing on, or otherwise selecting, a particular case window 156. One example of the type of additional information that server 150 causes to be displayed is shown in FIG. 10. FIG. 10 illustrates an example of an active case overview screen 190 that provides a plurality of details about a particular thermal therapy session that is currently active. More specifically, active case overview screen 190 includes a therapy graph 192, a plurality of phase windows 194, a therapy selector 196, an alarms selector 198, a notes selector 200, and a therapy change control 202.

Therapy graph 192 includes an X-axis that corresponds to time and at least one Y-axis that corresponds to temperature. Additional Y-axes may be provided that indicate additional parameters, such as flow rate, machine power, heat transfer, etc. Graph 192 includes a water temperature plot 204, a patient temperature plot 206, and a target patient temperature plot 208. In the example shown in FIG. 10, graph 192 also includes a machine power plot 210. Water temperature plot 204 shows the measurements of the fluid within thermal control unit 22 over time. Such water temperature measurements may be carried out by temperature sensor 56 and/or by another fluid temperature sensor 56. Controller 60 is configured to forward these temperature sensor readings to server 150 repetitively during a thermal therapy session.

Patient temperature plot 208 shows the measurements of the patient's temperature during the thermal therapy sessions. These temperature measurements come from patient temperature probe (86), which feeds these temperature measurements to thermal control unit 22. Controller 60 then repetitively forwards these patient temperature measurements to server 50 during the thermal therapy session. Target patient temperature plot 210 shows the desired temperature of the patient. The target patient temperature plot 210 comes from the preset 106 and/or is input manually by a user into thermal control unit 22 and forwarded to server 150. In those phases where the patient's temperature is being adjusted to a target temperature (as opposed to being maintained at a target temperature), server 150 may display target temperature plot 210 as an angled line that corresponds to the desired rate of heating or cooling that the user has entered (or defined via a preset) for that particular phase of the thermal therapy session. Machine power plot 210 refers to the amount of power the thermal control unit 22 is using at any given time to heat or cool the patient. Controller 60 repetitively determines these power levels and forwards them to server 150 during a particular thermal therapy session.

Graph 192 also includes a set of alarm bars 212 that, in the example of screen 190 shown in FIG. 10, are positioned along the top of graph 192. Alarm bars 212 have a length that corresponds to the amount of time that that particular alarm lasted. Alarm bars 212 are also placed on graph 192 at locations along the X-axis that correspond to the time at which the alarm commenced and the time at which the alarm ceased. Thus, the length of alarm bars 212 is directly proportional to the amount of time that the alarm has lasted. It will be understood that, in some embodiments, server 150 may be configured to display other parameters using bars similar to the alarm bars. For example, power bars may be displayed along the X-axis of graph 192 that show time periods when the power drawn by thermal control unit 22 exceeds, or is less than, one or more thresholds. Other types of bars, such as bars indicating cooling or heating, or still other parameters, may also or alternatively be included on graph 192.

Overview screen 190 (FIG. 10) also includes phase windows 194. Phase windows 194 correspond to the phases of the thermal therapy session that is graphed in graph 192. The number of phase windows 194 that are displayed for a given thermal therapy session may therefor vary from session to session. Each phase window 194 includes an identifier indicating high level information about that phase, such as heating, cooling, maintaining, etc. If a user pushes on, or otherwise selects, one of the phase windows 194, server 150 is configured to display additional information about that phase, such as, for example, a temperature graph 192 that shows only time period corresponding to that particular phase. Other detailed information about the phase may also, or alternatively, be displayed.

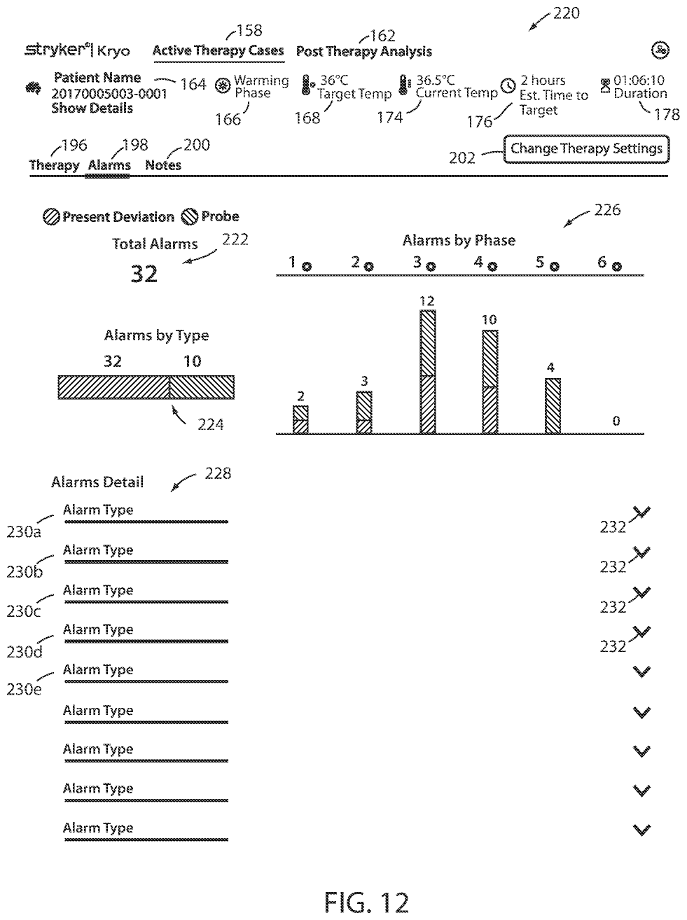

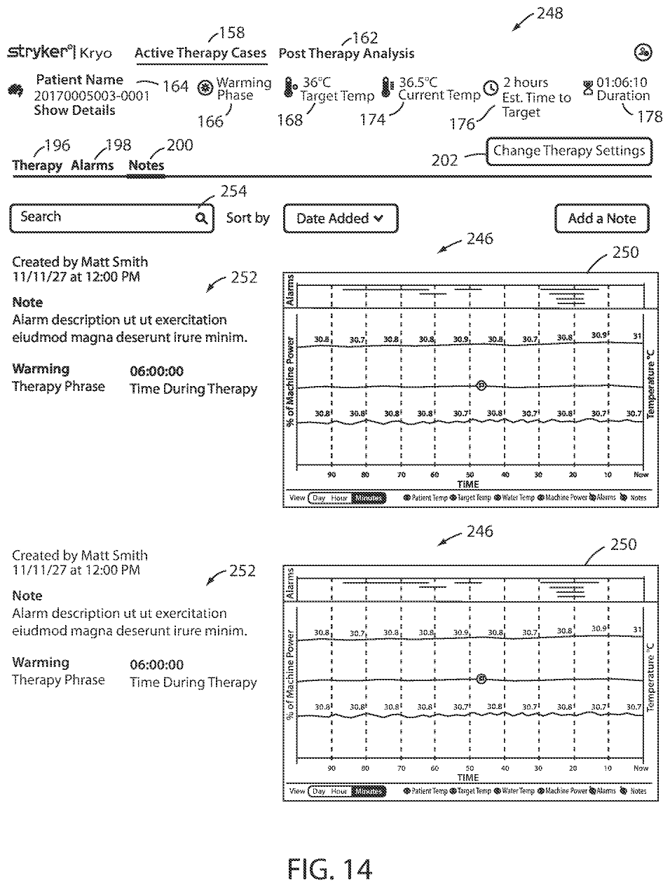

Therapy selector 196, alarm selector 198, and notes selector 200 of overview screen 190 (FIG. 10) allow a user to select different types of information that will be displayed by system 20 for a given thermal therapy session. These selectors 196, 198, and 200 also indicate what information is currently being displayed (such as through underlining of the particular indicator that is currently active). If the user selects the therapy selector 196, server 150 displays information about a currently active thermal therapy session, such as what is shown in FIG. 10. If the user selects alarms selector 198, server 150 displays an alarm overview screen, such as the one shown in FIG. 12, that provides additional information about the alarms associated with a particular active thermal therapy session. If the user selects note selector 200, server 150 displays a note overview screen, such as the note screen shown in FIG. 14, server 150 provides additional information about any notes that are associated with a particular active thermal therapy session.

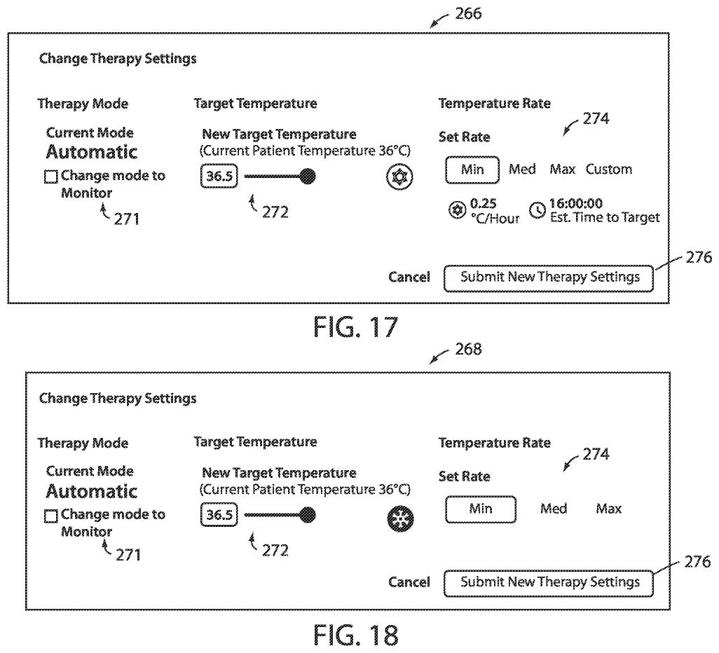

Therapy settings control 202, when activated by a user, allows the user to use his or her user device 170 to remotely change one or more settings on thermal control unit 22 for an active thermal therapy session. When a user activates thermal settings control 202, server 150 may be configured to display a remote control screen such as the remote control screen shown in FIG. 17.

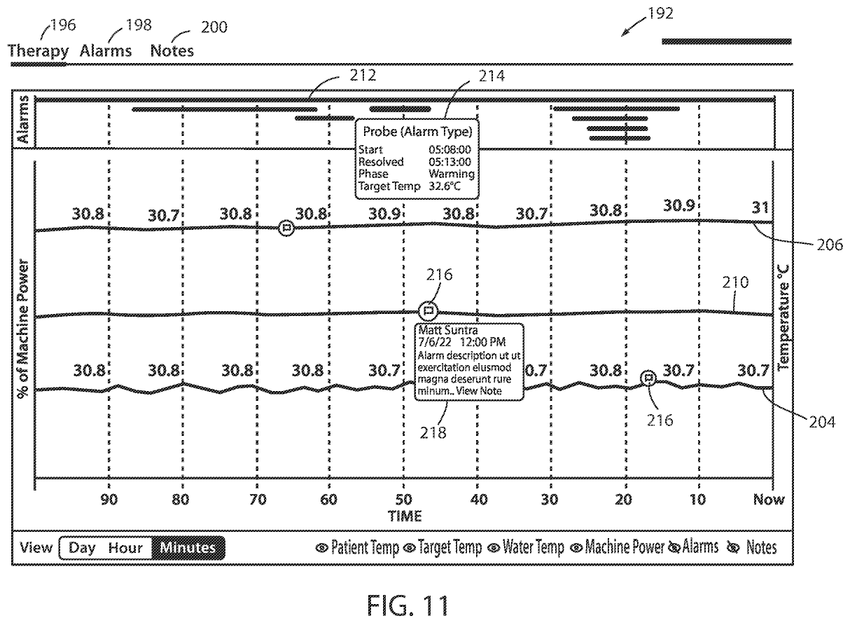

FIG. 11 illustrates a close up view of thermal graph 192. As shown therein, a user may click on (or otherwise select) any of the alarm bars 212. In response thereto, server 150 causes the user device 170 to display an alarm window 214 that provides additional information about the alarm associated with that particular alarm bar 212. Server 150 is also configured to display note icons 216 at selected locations on any of plots 204, 206, 208, and/or 210. The locations correspond to the content of the note. When the user clicks on, or otherwise selects, a particular note icon 216, a note window 218 is displayed that includes additional information about that particular note. In this manner, a user can append notes to graph 192 that relate to various aspects of a particular thermal therapy session. In some embodiments, server 150 forwards these notes to EMR server 124 for recording in a patient's medical records. In other embodiments, server 150 retains these notes separately from the patient's EMR records.

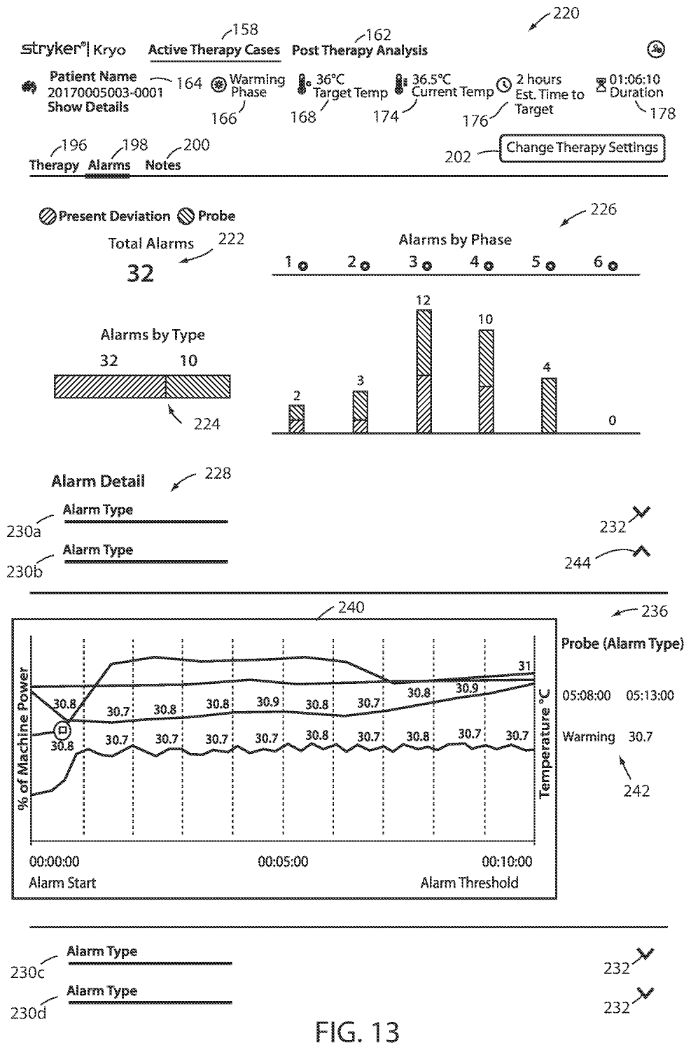

FIG. 12 illustrates an example of an alarm overview screen 220 that may be displayed on a user's device 170 in response to the user selecting alarm selector 198. Alarm overview screen 220 includes an alarm total 222, an alarm type indicator 224, an alarm bar graph 226, and an alarm listing 228. Alarm total 222 lists the total number of alarms for a particular thermal therapy session. Alarm type indicator 224 illustrates the total number of different types of alarms for a particular thermal therapy session. Alarm bar graph 226 illustrates the number of alarms, as well as type, for each phase of a thermal therapy session. Alarm listing 228 provides a listing of alarms 230 that occurred during a particular thermal therapy session. Screen 220 provides a brief overview of each alarm 230 in listing 228. If a user wishes to have more information displayed about a given alarm, he or she clicks on an expansion control 232 that is associated with each alarm 230. In response to the user clicking on expansion control 232, server 150 is configured to cause the user device 170 to display an alarm window 236 of the type shown in FIG. 13.

FIG. 13 illustrates alarm overview screen 220 that may be displayed on a user device 170 after a user has selected the expansion control 232 of alarm 230b. In response to selecting expansion control 232 of alarm 230b, server 150 causes the user device 170 to display alarm window 236. Alarm window 236 contains a graph segment 240 and alarm data 242. Graph segment 240 correspond to a segment of therapy graph 192 that contains the alarm 230b. In some embodiments, graph segment 240 contains a first portion of thermal graph 292 that precedes the occurrence of the particular alarm (e.g. alarm 230b) and a second portion of thermal graph 292 that succeeds the occurrence of the particular alarm. The segment of the graph that precedes the alarm may be a predetermined amount of time (e.g. ten minutes, thirty minutes, one hour, several hours, etc.) and the segment of the graph that occurs after the alarm may likewise be a predetermined amount of time (and which may be the same as, or different from, the predetermined amount of time that precedes the alarm). Graph segment 240 therefore provides a graph of the thermal therapy data for a selected time window around the time that an alarm occurred. This present the user with thermal therapy data that may be relevant to the particular alarm and/or that may help the user better understand the nature and/or severity off the alarm.

Alarm data 242 of alarm window 236 (FIG. 13) provides additional information about the corresponding alarm, such as the time at which the alarm started, the time at which the alarm was resolved, the phase in which the alarm occurred, and the target temperature for the patient at the time the alarm occurred. If the user wishes to close alarm window 236, he or she may click on, or otherwise select, a reduction control 244.

FIG. 14 illustrates an example of a note overview screen 248 that may be displayed on a user device 170 after a user has selected the note selector 200. Note overview screen 248 includes a plurality of note windows 246 and note searching field 254. Each note window 246 provides information about a particular note that was added during a particular thermal therapy session. In the example shown in FIG. 14, each note window 246 includes note data 252 and a note graph segment 250. Note data 252 displays the content of a particular note, while note graph segment 250 displays a segment of thermal graph 192 that corresponds to the time period that the note pertains to. As with alarm graph segment 240, note graph segment 250 may show a predetermined portion of graph 192 that occurs both immediately before and immediately after the time associated with the note. Note searching field 254 allows the user to search for keywords, or other information, regarding the notes associated with a particular thermal therapy session.

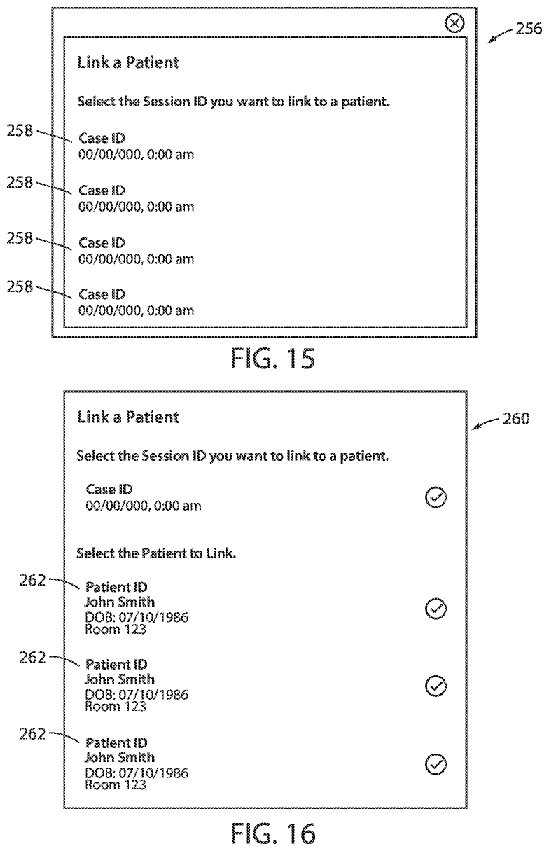

FIG. 15 illustrates a case selector screen 256 that displays a plurality of cases 258. Each case refers to a particular thermal therapy session. Case selector screen 256 allows a user to select a particular thermal therapy session (i.e. case 256). FIG. 16 illustrates a patient selector screen 260 that display a plurality of patients 262. Patient selector screen 256 allows the user to select a particular patient that should be associated with the particular thermal therapy session that was selected on screen 256 of FIG. 15. Once the user has selected a particular thermal therapy session and a particular patient using screens 256 and 260, server 150 is configured to associate the selected thermal therapy session with the selected patient. Screens 256 and 260 therefore allow a user to manually associate thermal therapy sessions with particular patients.

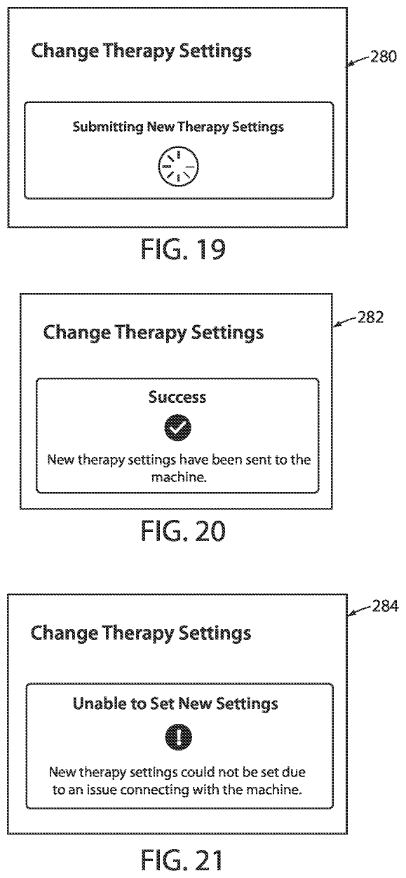

FIGS. 17 and 18 illustrate remote control screens that may be displayed on a user device in response to the user selecting the change therapy control 202 (see, e.g. FIG. 14). More specifically, FIG. 17 illustrates a warming remote control screen 266 and FIG. 18 illustrate a cooling remote control screen 268. Both screens 266 and 268 include a mode selector 271 that allows the user to remotely change the mode of operation of thermal control unit 22, a patient target temperature selector 272 that allows the user to remotely change the patient target temperature that thermal control unit 22 is trying to achieve, and a rate selector 274 that allows the user to remotely change the rate at which thermal control unit 22 attempts to change the patient's temperature. Screens 266 and 268 also include a submit control 276 that, when activated, causes server 150 to send a command to the corresponding thermal control unit 22 to implement the changes made by the user using screens 266 and/or 268. Server 150 is configured to cause the user device 170 to display screen 280 (FIG. 19) while the changes are being sent to thermal control unit 22. The user device 170 also displays screen 282 (FIG. 21) if the changes are successfully received by the thermal control unit 22, or screen 284 (FIG. 22) if the changes are not successfully received by the thermal control unit 22.

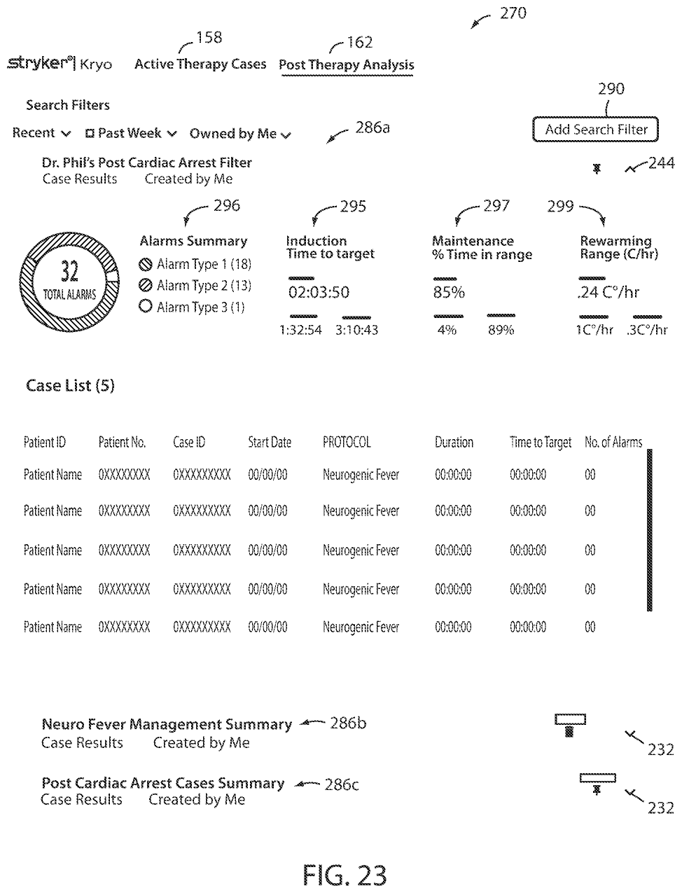

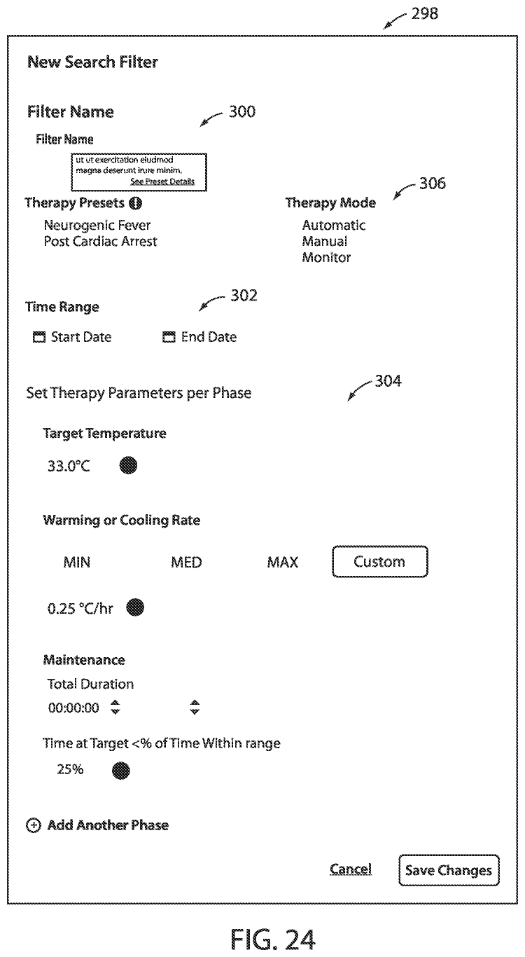

FIG. 22 illustrates an example of a post-therapy overview screen 270 that may be displayed to users having a particular role (e.g. nurse manager, doctor, administrator, etc.). Screen 270 includes a plurality of filter windows 286, a case listing 288, and a new filter control 290. Filter windows 286 correspond to filters that users of system 20 have set up for a particular healthcare facility. When a user selects a corresponding expansion control 232 on a particular filter window 286, server 150 is configured to expand the window 286 and display additional information about that particular filter. An example of this additional information in shown in FIG. 23. Case listings 288 correspond to all of the thermal therapy sessions that have been carried out by the thermal control units 22 within that particular healthcare facility. New filter control 290 allows a user to create a new filter. When the user selects new filter control 290, server 150 is configured to new filter screen such as that shown in FIG. 24.

FIG. 23 illustrates post-therapy overview screen 270 after a user has activated the expansion control 232 for filter window 286a. In response to this activation, server 150 causes user device 170 to display an expanded window 286a that includes additional information about the corresponding filter. As shown in FIG. 23, this additional information includes an alarm summary 296 for the filtered cases, an average induction time 295 for the filtered cases, an average maintenance range 297 for the filter cases, and an average re-warming rate 299 for the filtered cases. Other statistics may also or alternatively be displayed in expanded window 286a. Window 286a may also display individual cases that are part of that particular filter. Selecting those individual cases may bring up additional information about those cases. If the user selects the reduction control 244, server 150 is configured to instruct the user device 170 to reduce the size of window 286a back to the size shown in FIG. 22.

FIG. 24 illustrates a filter creation screen 298 that may be displayed in response to the user selecting the new filter control 290 (FIGS. 22-23). Filter creation screen 298 allows a user to create a new filter. After the new filter is created, it will be displayed in a filter window 286 on screen 270 (FIG. 23). Filter creation screen includes a name selection field 300, a time range selector 302, a parameter selector 304 section, and a mode selector 306—all of which can be used by the user to create a customized filter.

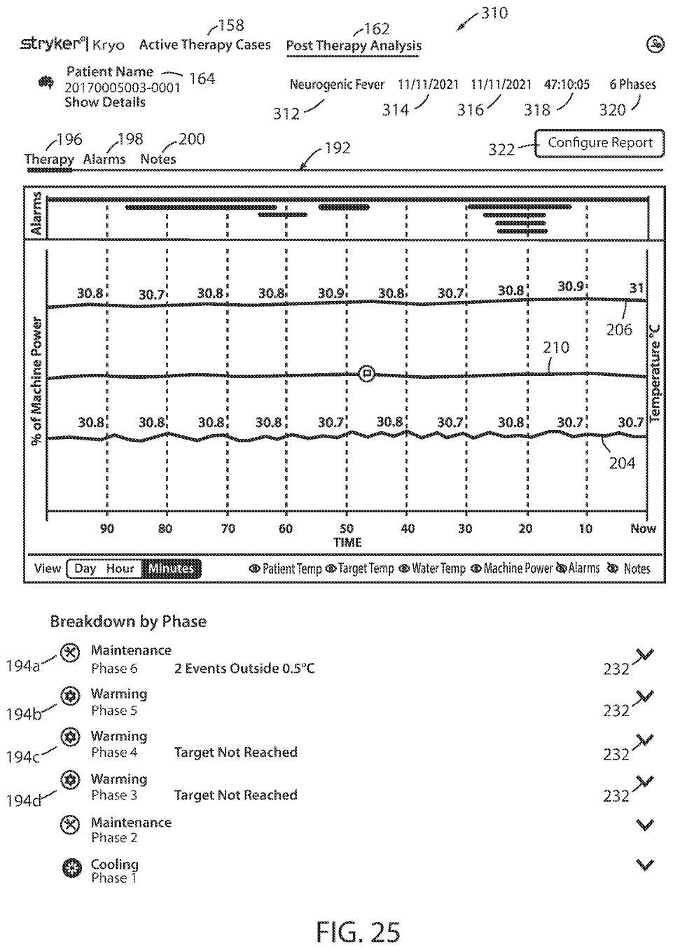

FIG. 25 illustrates a post-therapy individual case screen 310 that may be displayed to selected users after a thermal therapy session has been completed. Post-therapy screen 310 includes similar information as found in active care overview screen 190 (FIG. 10), and those items that are common to screen 190 include the same reference number and will not be described further herein. Screen 310 is displayed after a thermal therapy session is completed, while screen 190 is displayed while a thermal therapy session is active. In addition, screens 190 and 310 may be available to different users, depending upon their roles.

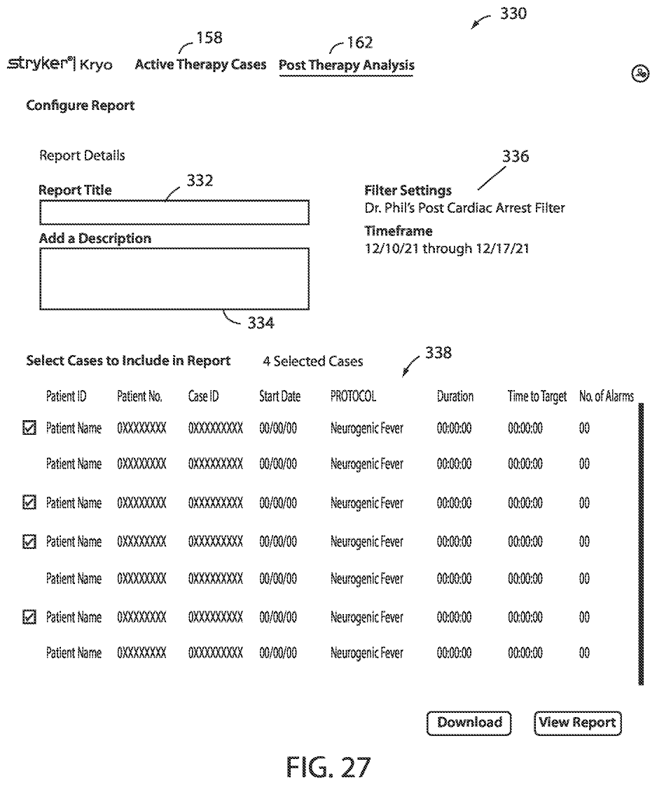

Screen 310 includes several items not found in screen 190. These include a filter identifier 312, a start date 314, and end date 316, a total duration indicator 318, a total phase indicator 320, and a report generator 322. If the user selects the report generator 322, server 150 is configured to cause the user device 170 to display a report generation screen, such as that shown in FIG. 27. As with screen 190, if a user presses on any of the expansion controls 232 of phase windows 194, server 150 is configured to enlarge the window 194 to display additional information about that particular phase.

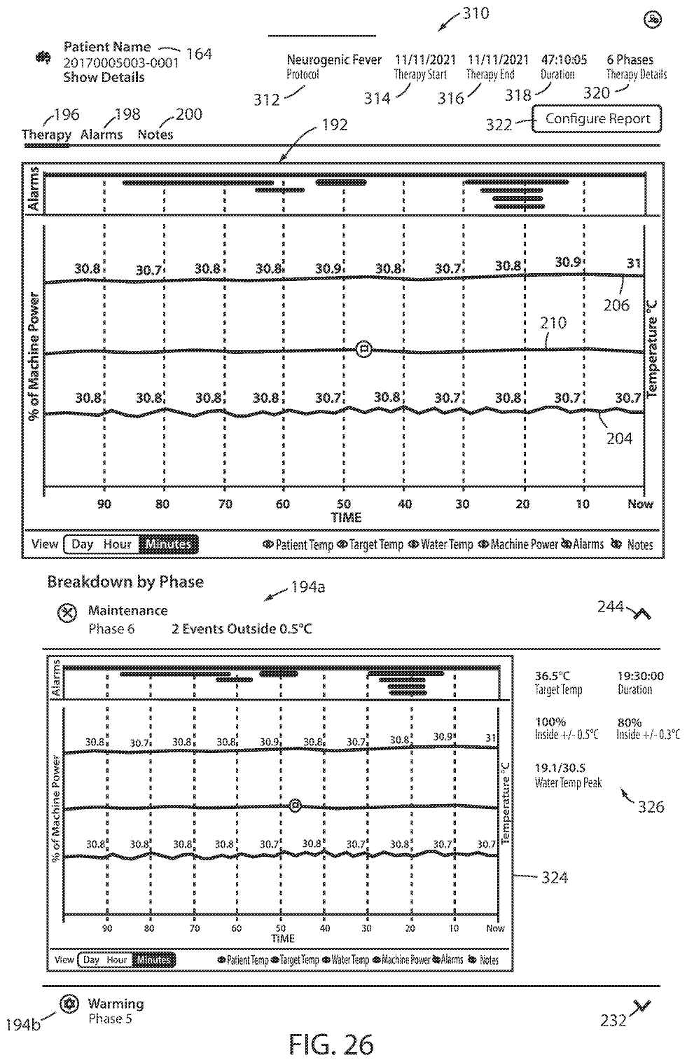

One example of such an enlarged window 194 is shown in FIG. 26, which displays screen 310 after the user has selected the expansion control 232 corresponding to phase window 194a. The enlarged window 194a includes a phase segment graph 324 and various phase data 326. The phase segment graph 324 corresponds to the segment of thermal graph 192 for the particular phase 194a. That is, phase segment graph 324 shows the portion of graph 192 that corresponds to the particular therapy phase of window 194a. Phase data 326 may include a variety of different information about the particular phase, such as, but not limited to, the patient target temperature, the phase duration, power level information, accuracy information to the target, peak fluid temperature, etc.

As noted, FIG. 27 illustrates a report configuration screen 330 that may be displayed in response to a user selecting the report generator 322 (see FIGS. 25 and 26). Report configuration screen 330 allows a user to generate a report corresponding to specific cases selected by the user. Report configuration screen 330 includes a title field 332 for entering a title of the report, a description field 334 for entering a description of the report, a report configuration window 336 for configuring additional information about the report, and a case selection window 338 for selecting individual cases to be included within the report. Once a report has been generated, server 150 is able to export the report in different formats, print the report, save the report, and/or perform other actions with the report.

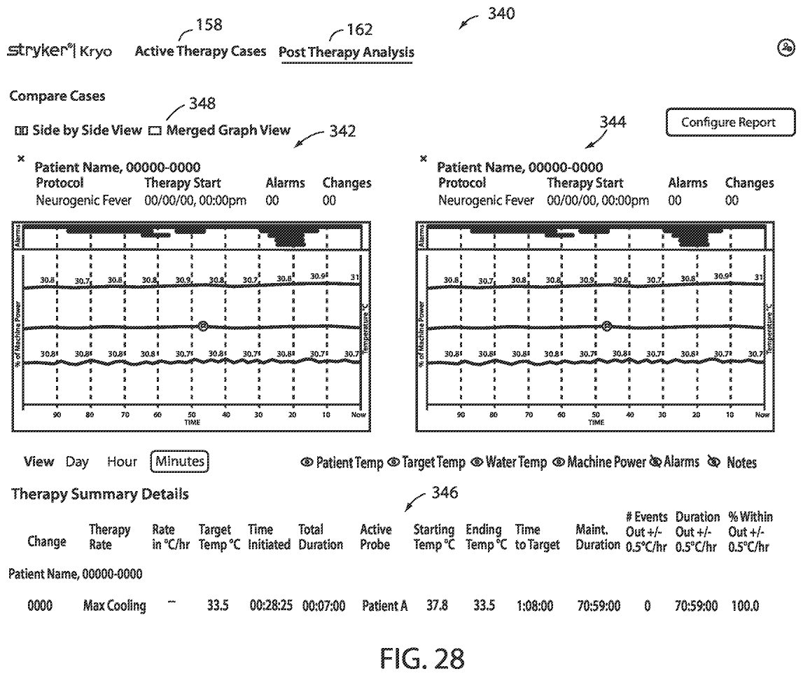

FIG. 28 illustrates a comparison screen 340 that may be displayed by system 20 on a user device 170 that compares selected thermal therapy sessions to each other in a side-by-side fashion. Comparison screen 340 includes a first case window 342, a second case window 344, a detailed summary window 346, and a merged view selector 348. First case window provides 342 information about a first selected thermal therapy session, while second case window 344 provides information about a second selected thermal therapy session. Detailed summary window provides additional details about the selected cases. Merge view selector 348, when selected, combines the data from both cases onto a single graph, instead of the side-by-side graphs shown in FIG. 28.

FIG. 29 illustrates an alarm selection screen 350 that allows an administrator of a healthcare facility, or other authorized user with the proper role, to select what alarms are going to be issued by system 20. Alarm selection screen 350 includes a plurality of alarms 352 and alarm switches 354 that allow the user to turn on or off each the alarm identified in the corresponding window. Alarm selection screen 350 also includes a site settings indicator 356 which indicates that screen 350 is a screen that is only visible to users who have a role that allows them to change configuration settings of system 20 for a particular healthcare facility.

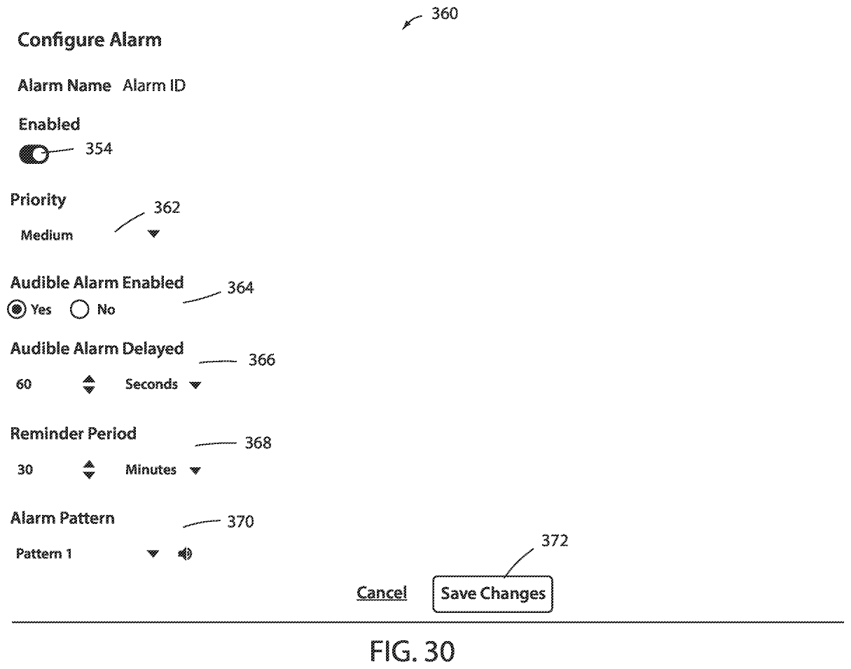

FIG. 30 illustrates an alarm configuration screen 360 that may be displayed in response to a user selecting a particular alarm 352 on screen 350 of FIG. 29. Alarm configuration screen 360 allows an authorized user to configure characteristics of individual alarms. Alarm configuration screen 360 includes a priority selector 362, an audible selector 364, a delay selector 366, a reminder period 368, and an alarm pattern selector 370. After the user has made any changes to a particular alarm, he or she presses on the alarm save control 372 and server 150 saves the alarm configurations in its memory.

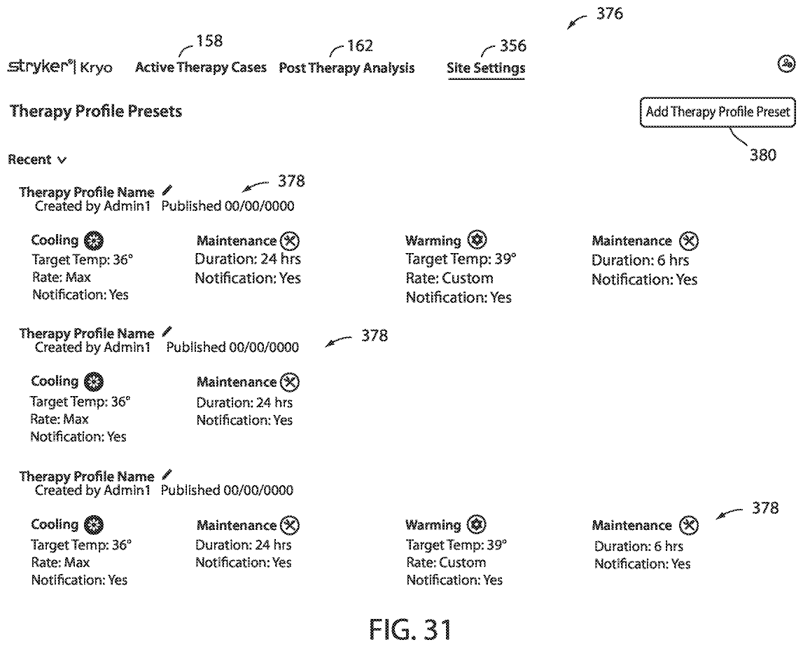

FIG. 31 illustrates a therapy preset overview screen 376 that may be displayed in order to inform users about the presets 106 (FIG. 5) that are currently defined for thermal control units 22 and/or to modify those presets 106. Preset overview screen 376 includes a plurality of preset windows 378 and a preset configuration control 380. Overview screen 376 displays a preset window 378 for each thermal preset 106 that is installed on any of the thermal control units 22 within the corresponding healthcare facility, and/or that are saved in a memory of server 150 but not yet installed on a thermal control unit 22. Each window 378 includes information defining that particular preset. In some embodiments, each window 378 may include an expansion control 232 that, when activated, displays an expanded window 378 that displays more information about the selected thermal preset.

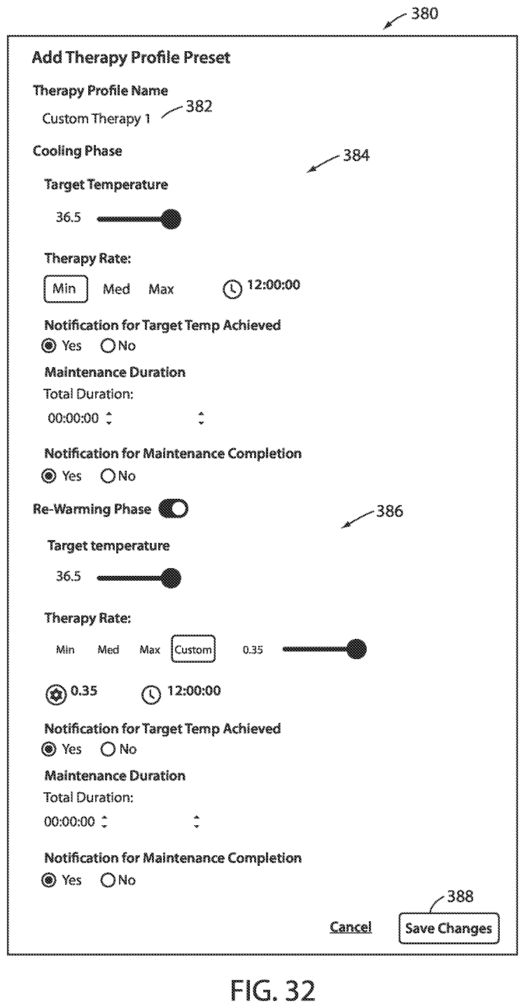

If a user selects the preset configuration control 380 (FIG. 31), server 150 is configured to display a preset definition screen, such as the preset definition screen 380 of FIG. 32. Preset definition screen 380 includes a preset name field 382, a cooling phase configuration window 384, a re-warming phase window 386, and a save changes control 388. Title field 382 allows the user to assign a title to the preset 106. Cooling phase window 384 allows the user to define characteristics of the cooling phase, or phases, or a particular preset 106. Re-warming phase window 386 allows the user to define characteristics of the re-warming phase, or phases. Save changes control 388 causes server 150 to save the characteristics of the preset 106 that were defined using preset definition screen 380.

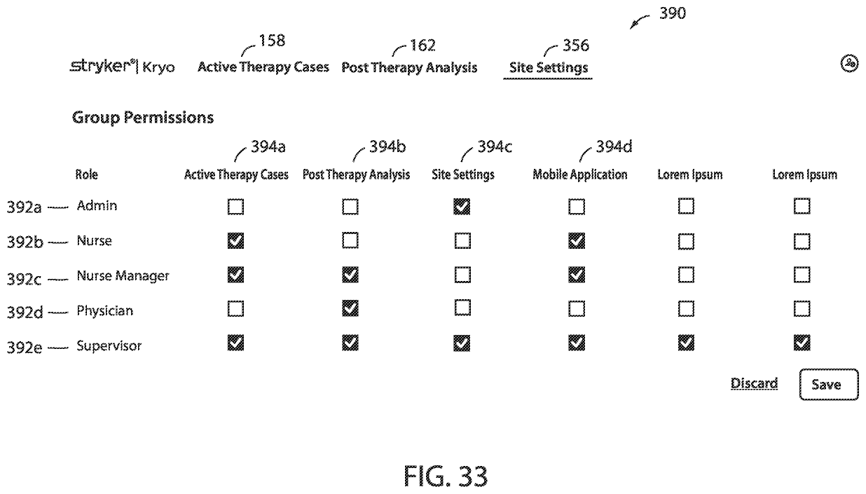

FIG. 33 illustrates a role permission screen 390 that allows an authorized administrator of a particular thermal control system 20 at a particular healthcare facility (or groups of facilities) to configure access levels for different users of system 20. In the example shown in FIG. 33, there are five different roles for system 20: an administrator role 392a, a nurse role 392b, a nurse manager role 392c, a physician role 392d, and a super user 392e. For each role, the user is able to select corresponding access privileges to system 20. More specifically, screen 390 includes a plurality of access settings 394a-d. Access setting 394a allows a user to view and utilize those screens and functions associated with active therapy cases (e.g. those screens shown in FIGS. 8-21); access setting 394b allows a user to view and utilize those screens and functions associated with post-therapy analysis (e.g. those screens shown in FIGS. 22-28); access setting 394c allows a user to view and utilize those screens and functions associated with the site settings (e.g. those screens shown in FIGS. 29-33); and access setting 394d allows a user to view and utilize system 20 using a mobile device that includes the native software application discussed above (as opposed to the web-based access, also discussed previously). Check boxes are associated with each role 392 and each access setting 394a, thereby allowing an authorized user to control access levels for different users associated with a particular healthcare facility.

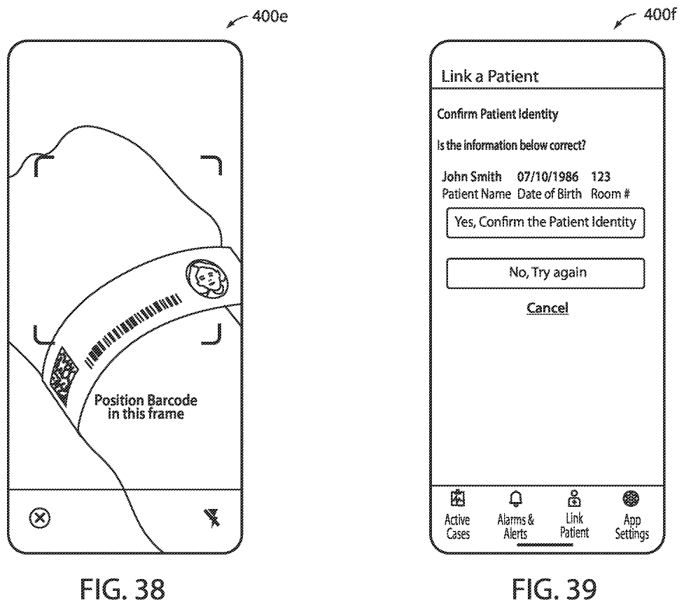

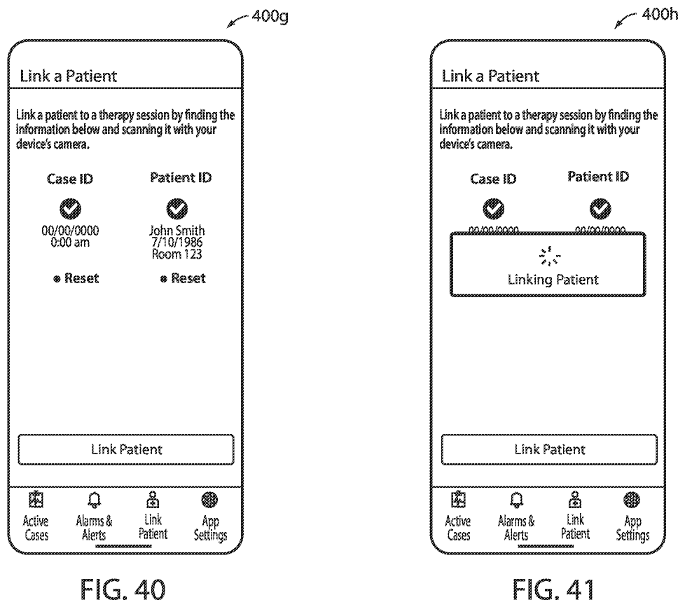

FIGS. 34-43 illustrate a plurality of screens 400a-j that may be displayed on a user device 170 when the user wishes to use a conventional camera and/or QR code reader built into the user device 170 for associating a particular thermal therapy session with a particular patient. Screens 400a-j may be displayed when user device 170 is executing a native application for communicating with server 150 (as opposed to web browser-based communication), although it will be understood that screens 400a-j could, in some embodiments, be utilized with a web-based browser. To associate a particular patient with a particular thermal therapy session, the user may navigate to screen 400d (FIG. 37) to capture an image of the QR code 130 displayed on the control panel 76 of the thermal control unit 22. Thereafter, the user may capture an image of a patient ID code-such as a QR code, bar code, or other identifier(s) that is/are incorporated into the patient's bracelet or wristband-using screen 400e (FIG. 38). After that, the user uses screens 400f and 400g (FIGS. 39 and 40) to link the selected thermal therapy session with the selected patient. Screens 400h, 400i, and 400j (FIGS. 41-43) may be displayed while the linking process is taking place and to inform the user whether the linking process was successful or unsuccessful.

After capturing the patient's ID code from his/her wristband, bracelet, or other item, the user device 170 may be configured to forward this patient ID code to server 150 so that server 150 may determine the identity of the particular patient associated with that ID. Server 150 carries this out by consulting EMR server 124 and/or another server (or servers) on the healthcare network that store data correlating a particular patient's identity with their patient ID code. In some embodiments, server 150 is configured to forward the patient's identity (e.g. name and/or other identifying information) back to user device 170 (and/or thermal control unit 22) so that user device 170 (and/or thermal control unit 22) can display the patient's identity thereon, thereby providing the caregiver with confirmation that they have associated the correct patient with a particular thermal therapy session. Alternatively, server 150 may skip sending the patient's identity to the user device 170 (and/or thermal control unit 22), and simply use the patient's identity (or ID code) to determine which EMR record to write the subsequent thermal therapy session data to.

After capturing the QR code 130 displayed on display 88 of thermal control unit 22, the user device 170 is configured to forward this QR code, or an identifier derived from this QR code, to server 150. Server 150 then matches this QR code, or the identifier derived therefrom, with the patient's ID code (or patient identity), and thus knows which patient the ensuing thermal therapy session data should be associated with.

In some embodiments, after a user has defined a new preset 106 using preset definition screen 380 (FIG. 32), server 150 is configured to forward the preset 106 to one or more thermal control unit 22, but not have thermal control unit 22 automatically install the new preset 106. Instead, thermal control unit 22 may be configured to require a user to confirm the new preset and/or authorized the new preset before controller 60 will install the new preset.

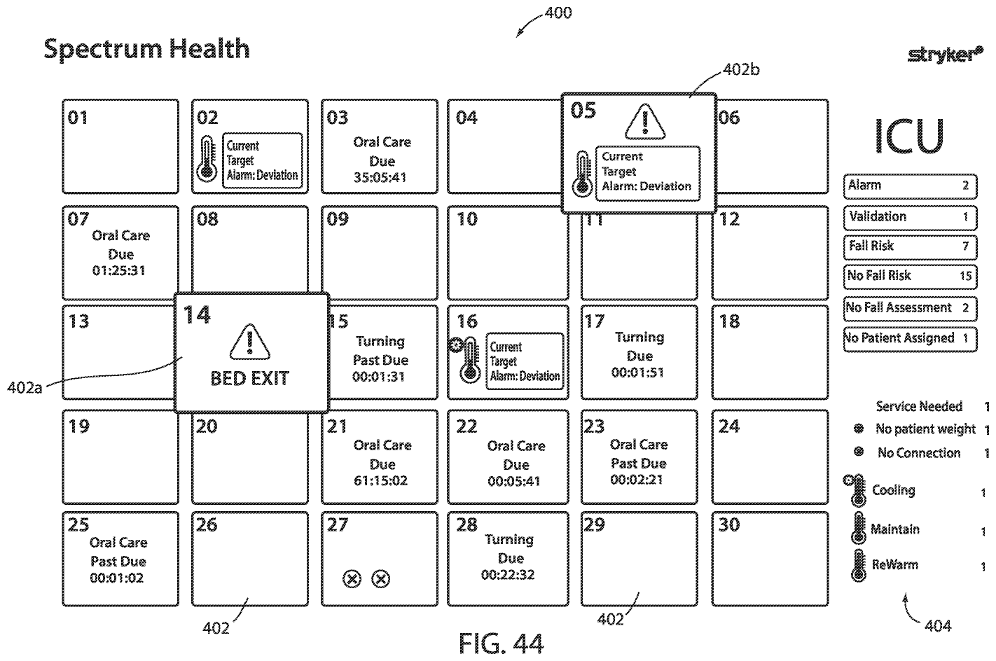

FIG. 44 illustrates an example of a dashboard screen 400 that may be displayed on any of user devices 170. Dashboard screen 400 combines data from thermal control units 22 with status data from patient support apparatuses 116. As shown therein, dashboard screen 400 includes a plurality of room icons 402 that correspond to different rooms within the healthcare facility. For each room icon 402, server 150 adds information about the patient support apparatus 116 and/or thermal control unit 22 that is being used in that room. Such information may include an exit alert, as detected by the patient support apparatus 116, one example of which is shown for room icon 402a. Such information may also include an alarms associated with thermal control unit 22, such as is shown for room icon 402b. Temperature management server 150 obtains the patient support apparatus data displayed on dashboard 400 by communicating with patient support apparatus server 160. Dashboard screen 400 may also include an overview window 404 that displays a summarized set of data regarding thermal control units 22 and/or patient support apparatuses 116.

It will be understood that any of the screens and/or functionality illustrated in FIGS. 7-33 herein may be alternatively displayed on display 88 of thermal control unit 22 and/or implemented into the control panel 76 of thermal control unit 22. In such embodiments, controller 60 is adapted to display these screens on display 88 and/or implement their functionality on control panel 76. Controller 60 is adapted to display these screens and execute their functionality either alone or in communication with one or more servers, including, but not limited to, servers 124, 160, and/or 160. As a result, any of the previously described functions that a user was able to carry out using a user device 170 can, in such modified embodiments, be carried out using control panel 76 of thermal control unit 22. In some embodiments, thermal control unit 22 is adapted to allow a user to carry out any of the functions described herein via both a user device 170 or via control panel 76.

In some embodiments, any of the functionality associated with FIGS. 34-43 may be built into thermal control unit 22. In such embodiments, thermal control unit 22 may include its own built-in bar code scanner, QR code scanner, and/or camera. The scanner or camera, in some such embodiments, may be part of a handheld device that is communicatively coupled to thermal control unit 22, such as via a USB cable, another type of cable, a Bluetooth connection, or another type of wireless connection. The user may then scan or take a picture of the patient's wristband, bracelet, or other item and capture the information stored thereon. Controller 60 may forward this information, or an identifier derived therefrom, to server 150 so that server 150 knows with what patient to associate the upcoming thermal therapy session data. In such embodiments, it is not necessary for thermal control unit 22 to display QR code 130 and have it captured by a user device 170. Instead, controller 60 generates a unique identifier for every thermal therapy session that it oversees and forwards that unique identifier to server 150 (along with the patient information captured from the patient's wristband, bracelet, or other identifying item). Server 150 is then able to match the thermal therapy session data with a particular patient.

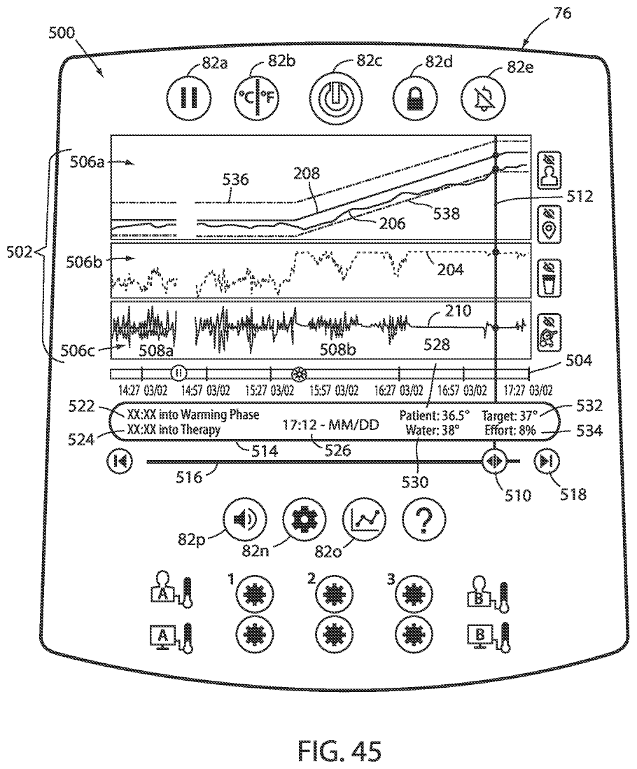

FIG. 45 illustrates a therapy session screen 500 that may be displayed by controller 60 on control panel 76 in some modified embodiments of thermal control unit 22. Therapy session screen 500 may be displayed during a thermal therapy session and/or it may be displayed after a thermal therapy session. Therapy session screen 500 includes a graph 502 having a horizontal time axis 504 and a plurality of segments 506a-c. Upper segment 506a graphs the patient temperature over time, as measured by one or more patient temperature probes 86. Middle segment 506b graphs the temperature of the circulating fluid (which may be water, or a water mixture) over time, as measured by fluid temperature sensor 56. Lower segment 506c graphs the amount of machine effort exerted by thermal control unit 22 over time, as measured by, for example, the amount of power consumed by heater 44 and/or chiller 42. The power graphs of lower segment 506c may be graphed in terms of absolute power (e.g. watts, or other power units), or it may be graphed in terms of relative power-such as a percentage of the heater 44 and/or chiller 42's maximum power. Or it may be graphed in still other manners.

Time axis 504 extends horizontally underneath segments 506a-c and indicates the corresponding time for all three of the segments 506a-c. Time axis 504 may also include a plurality of event icons 508 positioned thereon that correspond to times at which an event took place during the thermal therapy session. In the example shown in FIG. 45, there are two time event icons 508a and 508b. First event icon 508a corresponds to the moment that a user pressed the pause control 82a and temporarily paused the thermal therapy session. This pause is reflected in the segments 506a-c where no patient temperature readings 206 are graphed in upper segment 506a, no fluid temperature readings 204 are graphed in middle segment 506b, and no machine effort readings 210 are graphed in lower segment 506c. In some embodiments, controller 60 may be modified to continue to display patient temperature readings 206 in upper segment 506a and/or fluid temperature readings 204 in middle segments 506b while the thermal therapy session is paused (but omit machine effort readings 210 in lower segment 506c or simply display a zero in lower segment 506c while therapy is paused).

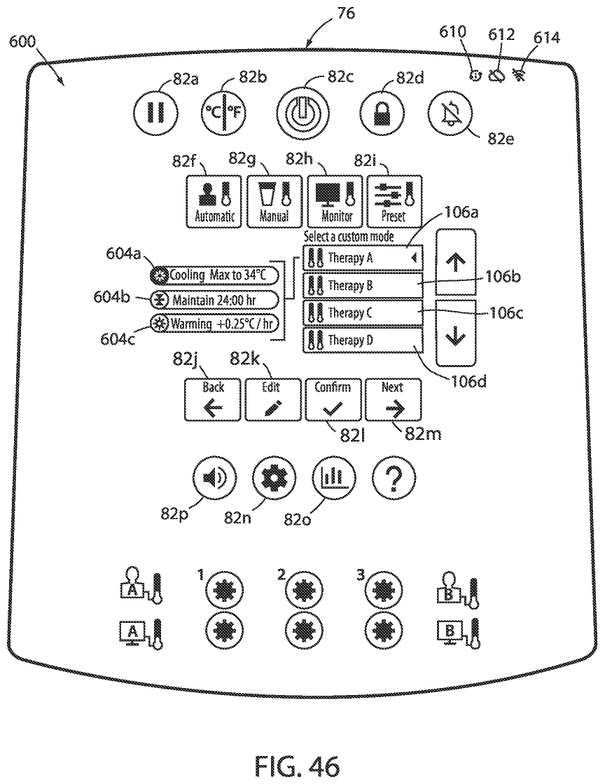

Second time event icon 508b corresponds to the time at which thermal control unit 22 switched to the warming phase of the thermal therapy session (from a previous maintenance phase). In some embodiments, controller 60 is configured to display an event icon any time thermal control unit 22 switches from one phase to another. In some embodiments, there are three potential phases in which thermal control unit 22 may operate: a warming phase in which the patient's temperature is warmed to a target temperature, a cooling phase in which the patient's temperature is cooled to a target temperature, and a maintenance phase in which the patient's temperature is maintained at a target temperature. In some embodiments, controller 60 is configured to automatically add an event icon 508 to the time axis 504 of graph 502 at a location corresponding to the time at which thermal control unit 22 switches from one phase to another. In some embodiments, controller 60 adds an event icon 508 resembling a snowflake when a cooling phase begins; adds an event icon 508 resembling the sun when a warming phase begins, and adds an event icon 508 comprising a dual up/down set of arrows (as shown in FIG. 46) when a maintenance phase begins. Other symbols may, of course, be used.

In some embodiments, controller 60 is configured to automatically add event icons 508 to time axis 504 (FIG. 45). In other embodiments, controller 60 may be modified to allow a user to manually add event icons 508, either in addition to, or in lieu of, controller 60's automatic addition of event icons 508. In some embodiments, controller 60 is configured to automatically add an event icon 508 for any of the following events that it detects: the starting of a new phase, the pausing of therapy (using control 82a), the detection of an alarm, and/or other events. In some such embodiments, controller 60 is configured to allow a user to customize what event icons 508 are automatically added to time axis 504, including selecting which alarms (if any) are to be shown on time axis 504 with event icons 508 and which alarms (if any) are not to be shown on time axis 504.

Therapy session screen 500 (FIG. 45) further includes a cursor 510, a vertical bar 512 coupled to cursor 510, an information oval 514, a horizontal bar 516, a skip forward control 518, a skip backward control 520, and a volume control 82p. Cursor 510 is slidable horizontally along horizontal bar 516. A user simply presses on cursor 510 and slides his or her finger horizontally along horizontal bar 516. Controller 60 responds to the user's sliding of his or her finger by sliding cursor 510 along horizontal bar 516 in a manner that matches position of the user's finger. In addition, controller 60 responds by shifting vertical bar 512 to the position that the user has slid cursor 510. In other words, controller displays cursor 510 and vertical bar 512 such that they appear attached to each other. In this respect, as cursor 510 is slid left or right, vertical bar 512 slides left or right with cursor 510. When the cursor 510 reaches either the left-most or right-most position on therapy session screen 500, controller 60 automatically starts scrolling to reveal additional data that is currently off-screen. In other words, if the user moves cursor 510 all the way to the left, controller 60 will automatically start to scroll further towards the left, revealing any additional data that is currently not viewable on screen 500. Similarly, if the user moves cursor 510 all the way to the right, controller 60 will automatically start to scroll further towards the right, revealing any additional data that is currently not viewable on screen 500.

Vertical bar 512 intersects time axis 504 and upper, middle, and lower segments 506a-c at different locations of graph 502, depending upon where the user has moved cursor 510 to. Controller 60 displays data in information oval 514 that corresponds to the time position in which vertical bar 512 is currently positioned. In other words, if vertical bar is slid to a time corresponding to seventeen hours and twelve minutes into the therapy session (as shown in FIG. 45), controller 60 will display in information oval 514 data that corresponds to that particular time during the therapy session. However, if the user, for example, slides cursor 510 to the time along axis 504 corresponding to, say fourteen hours and fifty-seven minutes, controller 60 will change the information shown in information oval 514 to match the data that was measured at fourteen hours and fifty-seven minutes into the therapy session. Controller 60 therefore updates the information shown in information oval 514 according to where vertical bar 512 intersects time axis 504. In some embodiments, controller 60 updates the information in information oval 514 in substantially real time such that there is no visible delay between bar 512 being repositioned and the information within information oval 514 being updated.

As shown in FIG. 45, information oval 514 includes a phase time indicator 522, a therapy time indicator 524, a centered time indicator 526, a patient temperature indicator 528, a fluid (water) temperature indicator 530, a patient target temperature indicator 532, and a machine effort indicator 534. Phase time indicator 522 indicates the amount of time between the moment represented by the intersection of vertical bar 512 and the beginning of the particular thermal therapy phase in which the vertical bar 512 is positioned. In other words, if vertical bar 512 intersects time axis 504 anywhere in a region of graph 502 corresponding to a warming phase, indicator 522 displays the amount of time between the beginning of that warming phase and the current position of the vertical bar 512.

Therapy time indicator 524 (FIG. 45) indicates the amount of time that has elapsed since the moment the thermal therapy session began up to the moment along time axis 504 that is currently intersected by vertical bar 512. Centered time indicator 526 may indicate the total amount of time that has elapsed since the moment the thermal therapy session began up to the present moment (regardless of where vertical bar 512 is currently positioned). Thus, centered time indicator 526 displays the current total therapy time, regardless of where vertical bar 512 is currently positioned, while therapy time indicator 524 displays the total elapsed therapy time between the beginning of the therapy session and the time where vertical bar 512 currently intersects the time axis 504.

Patient temperature indicator 528 indicates the measured patient temperature at the moment in time indicated by vertical bar 512's intersection with time axis 504. Fluid temperature indicator 530 measures the temperature of the fluid delivered to outlets 58 at the moment in time indicated by vertical bar 512's intersection with time axis 504. Target temperature indicator 532 indicates the patient target temperature at the moment in time indicated by vertical bar 512's intersection with time axis 504. And machine effort indicator 534 indicates the power level, or other parameter indicative of chiller 42 and/or heater's 44's efforts, at the moment in time indicated by vertical bar 512's intersection with time axis 504.

When controller 60 detects that skip forward control 518 (FIG. 45) is pressed by a user, controller 60 moves forward in time and makes corresponding adjustments to the information that is shown in graph 502. When controller 60 detects that skip back control 520 (FIG. 45) is pressed by a user, controller 60 moves backward in time and makes corresponding adjustments to the information that is shown in graph 502. More specifically, controller 60 moves forward or backward to the next closest event on time axis 504 and recenters graphs 502 around that next closest event in time. In the example illustrated in FIG. 45, if a user presses the skip back control 520, controller 60 shifts graph 502 backwards in time so that the closest previous event marker (e.g. warming phase event icon 508b) is shown in the center of graph 502. If the user presses skip back control 520 a second time in the example of FIG. 45, controller 60 would shift graph 502 so that pause event icon 508a was moved to the center of graph 502. If the user then pressed the skip forward control 518, controller 60 would shift graph 502 such that it was no longer centered around pause event icon 508a, but instead was centered around warming phase event icon 508b. Thus, the skip forward 518 and skip backward 520 buttons move the information show in graph 502 forward and backward to the nearest event icon 508 on time axis 504. The user is therefore easily able to skip forward or backward to see the data of graph 502 in those moments surround the events indicated on time axis 504.

FIG. 45 further includes a maximum acceptable deviation marker 536 and a minimum acceptable deviation marker 538 in upper graph segment 506a. Both of these markers 536 and 538 are shown in FIG. 45 a dashed lines that defined around the patient's target temperature graphed in segment 506a. These markers 536 and 538 together define an acceptable range of the patient's temperature with respect to the target temperature for the patient. In some embodiments, the values of these markers 536 and/or 538 may be customized by the caregiver, while in other embodiments the values of markers 536 and/or 538 are fixed. In either embodiments, the values of markers 536 and 538 may define a range of acceptable temperatures that are within, say, 0.5 degrees Celsius of the patient's target temperature. Thus, in this particular example, maximum acceptable deviation marker 536 would be graphed one half of a degree Celsius above the patient's target temperature, and minimum acceptable deviation marker 538 would be graphed one half of a degree Celsius below the patient's target temperature. Markers 536 and 538 provide an easy reference for caregivers to see how well the patient's temperature is tracking the target temperature.

In some embodiments, controller 60 is configured to automatically take one or more actions whenever the patient's measured temperature deviates from the range defined between the maximum acceptable deviation marker 536 and the minimum acceptable deviation marker 538. In some embodiments, controller 60 automatically takes the action of adding an event icon 508 to time axis 504 when such a deviation from the temperature range occurs. In some embodiments, either in addition to, or in lieu of, adding an event icon 508, controller 60 is configured to issue an alert. The alert may be a local alert that is carried out solely at thermal control unit 22 (e.g. turning on a light, emitting a sound, and/or taking other steps), and/or the alert may be a remote alert, in which case controller 22 sends a message to one or more servers (e.g. 150), and those one or more servers may send a communication to one or more user devices 170. In those embodiments where controller 60 automatically adds an event icon 508 to time axis 504, controller 60 may be configured to also automatically add another event icon 508 to time axis 504 at the moment in time where the patient's temperature returns to being within the temperature range defined between markers 536 and 538.

In some embodiments of thermal control unit 22, controller 60 is configured to display the lower segment 506c of graph 502 in a multi-colored manner. Specifically, controller 60 may be configured to display the machine efforts that are exerted toward cooling in a different color than the machine efforts that are directed toward heating. In some such embodiments, controller 60 displays the cooling efforts in a blue shade and the heating efforts in an orange shade. In the graph shown in lower segment 605c of FIG. 45, the machine effort readings made for heating the circulating fluid are displayed as positive values and the machine effort readings made for cooling the circulating fluid are displayed as negative values. Thus, in at least one embodiments, those portions of the graph in lower segment 506c that have positive values are displayed with an orange shade and those portions of the graph in lower segment 506c that have negative values are displayed with a blue shade. This color coding provides a more intuitive understanding to the user of how the machine's efforts are being directed (i.e. toward heating or toward cooling).

Therapy session screen 500 (FIG. 45) also includes a volume control 82p that may be used to adjust the volume of aural sounds emitted by thermal control unit 22. In some instances, control 82p may act as a mute button that toggles between allowing sound to be emitted by thermal control unit 22 and shutting off any sound emitted by thermal control unit 22. In other embodiments, pressing and holding on control 82p may gradually change the sound level, depending upon how long the control 82p remains pressed. In such instances, controller 60 may be configured to gradually increase the sound level to a maximum, then drop to a minimum and start increasing the sound again, or vice versa, until the user lifts his or her finger from volume control 82p.

FIG. 46 illustrates a modified therapy preset selection screen 600 that may be displayed on display 76 of thermal control unit 22, in some embodiments. Therapy preset screen 600 of FIG. 46 is a modified version of the screen shown in FIG. 5. In some embodiments, thermal control unit 22 is configured to display a preset selection screen of the type shown in FIG. 5, while in other embodiments thermal control unit 22 is configured to display a preset selection screen 600 of the type shown in FIG. 46. Still other types of preset selection screens may, of course, be implemented by thermal control unit 22.

Preset selection screen 600 of FIG. 46 differs from the therapy preset screen of FIG. 5 primarily in that preset selection screen 600 includes a preset summary 602 for each of the preset therapies 106a-d. Each preset summary 602 includes a phase indicator 604 for each of the phases within that preset therapy 106. The phase indicators 604 are illustrated on screen 600 from top to bottom in the same sequence that they follow in the actual preset therapy 106. Thus, in the example shown in FIG. 46, therapy A starts with a cooling phase (indicator 604a), which is followed by a maintenance phase (indicator 604b), which is, in turn, followed by a warming phase (indicator 604c). In some embodiments, the phase indicators 604 are color coded according to their specific phase: cooling phase indicators 604a have a blue shade, maintenance phase indicators 604b have a green shade, and warming phase indicators 604c have an orange shade. The different colors may refer to the text displayed by the indicators, to the border surrounding the text, and/or to the background of the text shown in the indicators 604.

Each phase indicator 604 provides information about that particular phase, including both text and an icon indicating whether the phase is a warming phase, a cooling phase, or a maintenance phase. Each phase indicator 604 may also provides an indication of the time of the therapy phase, the effort to be exerted for a cooling phase, and/or the rate at which a patient is to be warmed for a warming phase. Controller 60 is configured to update the phase summary 602 and corresponding phase indicators 604 to match whichever preset therapy control 106a, 106b, 106c, and/or 106d that the user selects. The user is therefore provided with a visual summary of what each of the preset therapies 106a-d are configured to implement.

In some embodiments, preset selection screen 600 and/or one or more other screens include one or more status icons 610, 612, 614, etc. positioned in the upper right corner of control panel 76. In the example shown in FIG. 46, preset selection screen 600 includes a software update icon 610, a cloud connectivity icon 612, and a network connectivity icon 614. In some embodiments, controller 60 may also be configured to display other status icons, such as an icon indicating different languages that a user may select and which, when selected, cause controller 60 to display information on control panel 76 in a different language. Still other status icons may be included on screen 600.

Software update icon 610 is displayed by controller 60 on screen 600 when a software update is available for thermal control unit 22. Controller 60 displays software update icon 610 in response to receiving a message from either patient support apparatus server 160 and/or thermal management server 150 indicating that a software update is available for thermal control unit 22. Cloud connectivity icon 612 indicates whether thermal control unit 22 is successfully coupled to thermal management server 150 or not. In the example shown FIG. 46, cloud connectivity icon 612 includes a diagonal bar through a cloud, which indicates that thermal control unit 22 is not connected to thermal management server 150. When a successful connection between thermal control unit 22 and thermal management server 150 exists, controller 60 changes cloud connectivity icon 612 by removing the diagonal bar, in some embodiments. Network connectivity icon 614 indicates whether thermal control unit 22 is successfully connected to the healthcare facility's local network 122, either via a wireless connection (e.g. WiFi) or a wired connection (e.g. an Ethernet cable). In the example shown in FIG. 46, a diagonal bar through the network status icon 614 indicates there is currently no network connection. Controller 60 removes this diagonal bar when a network connection is successfully established.

In some embodiments, controller 60 is configured to display one or more of icons 610, 612, 614, etc. across multiple different screens. That is, if a user navigates to different screens displayed on control panel 76, controller 60 is configured to display status icons 610, 612, 614, etc. across multiple, if not all, of these screens. In some embodiments, controller 60 is configured to display one or more of the status icons 610, 612, 614, etc. on any of the screens, or partial screens, shown in FIGS. 4-33 and 45-52.

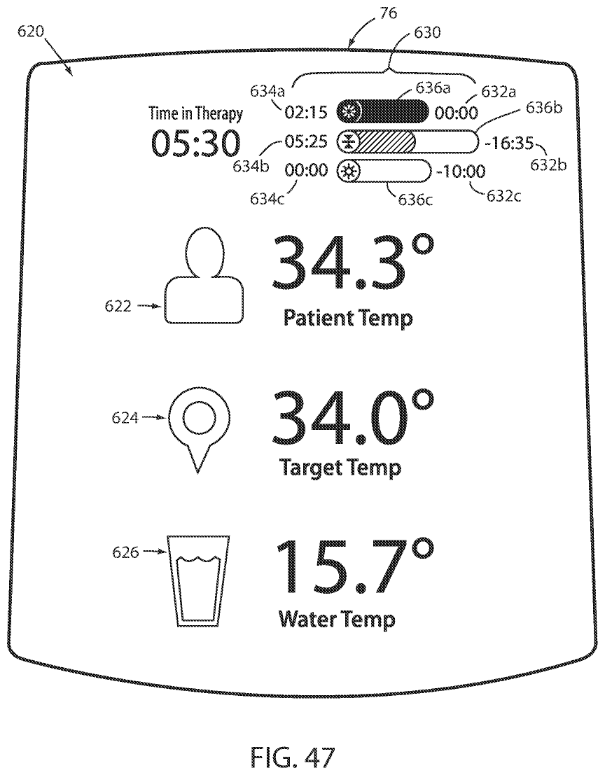

FIG. 47 illustrates screen saver screen 620 that may be displayed by thermal control unit 22 after controller 60 detects no user interaction with control panel 76 for a predetermined amount of time during a thermal therapy session. In some embodiments, the predetermined amount of time may be on the order of a one to several minutes, although other time periods may be utilized. Regardless of the specific time period utilized, controller 60 is configured to monitor usage of control panel 76. If controller 60 does not detect any user activity for the predetermined amount of time while a thermal therapy session is ongoing, controller 60 may be configured to display a screen like screen saver screen 620.

Screen saver screen 620 includes a patient temperature indicator 622, a patient target temperature indicator 624, a fluid temperature indicator 626, a time indicator 628 that indicates the amount of time that has elapsed since the current thermal therapy session began, and a preset summary indicator 630. Preset summary indicator 630 includes individual phase indicators 632 that are somewhat similar to phase indicators 604 in that they provide additional details about each phase of the current therapy session. However, unlike phase indicators 604, phase indicators 632 provide an indication of how far into the preset therapy 106 the current thermal therapy session has progressed. Specifically, each phase indicator 632 includes an elapsed time indicator 634, a therapy status oval 636, and a time-to-go indicator 638.

The elapsed time indicator 634 indicates how much time has been spent in the corresponding phase. Thus, in the example shown in FIG. 47, elapsed time indicator 634a shows that the cooling phase lasted two hours and fifteen minutes and that the maintenance phase has been ongoing for five hours and twenty five minutes, and that the warming phase has not yet begun. The therapy status oval 636 provides a visual indication of how far into each therapy phase the therapy session has begun. As time progresses for each therapy phase, controller 60 fills in more and more of the therapy oval until, as shown in the cooling phase indicator 632a, the entire therapy status oval 636a is filled in, indicating that the cooling phase has been completed. Only a portion of the maintenance phase has been completed in the example of FIG. 47, as indicated by only a portion of the therapy status oval 636b being filled in. And none of the therapy status oval 636c is filled in in the example of FIG. 47, thereby indicating that the cooling phase has not yet begun.

The time to go indicators 638 indicate how much time remains in each corresponding phase. Thus, in the example shown in FIG. 47, there is no time left in the cooling phase (which has been completed), there are sixteen hours and thirty-five minutes left in the maintenance phase (which is in progress), and there are ten hours left in the warming phase (which has not yet begun). As with the phase indicators 604 of FIG. 46, the phase indicators 632 of FIG. 47 may be color coded according to their phase, with cooling phase indicators 632a having a blue shade, maintenance phase indicators 632b having a green shade, and warming phase indicators 632c having an orange shade. The different colors may refer to the text (numbers) displayed by the indicators, to the border of therapy status ovals 636, and/or to the inside of the therapy status ovals 636.

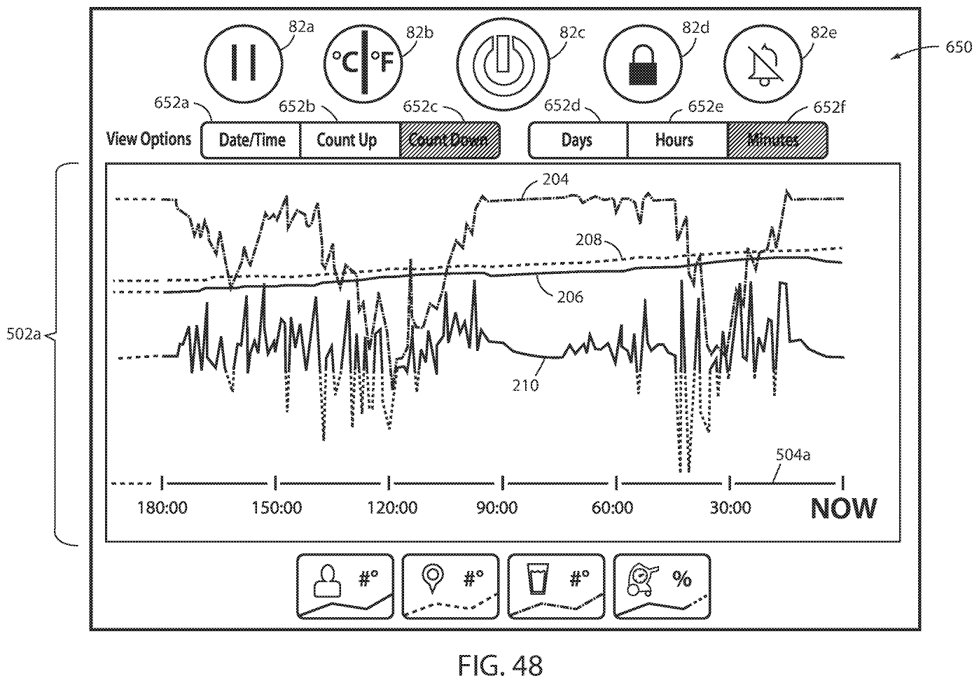

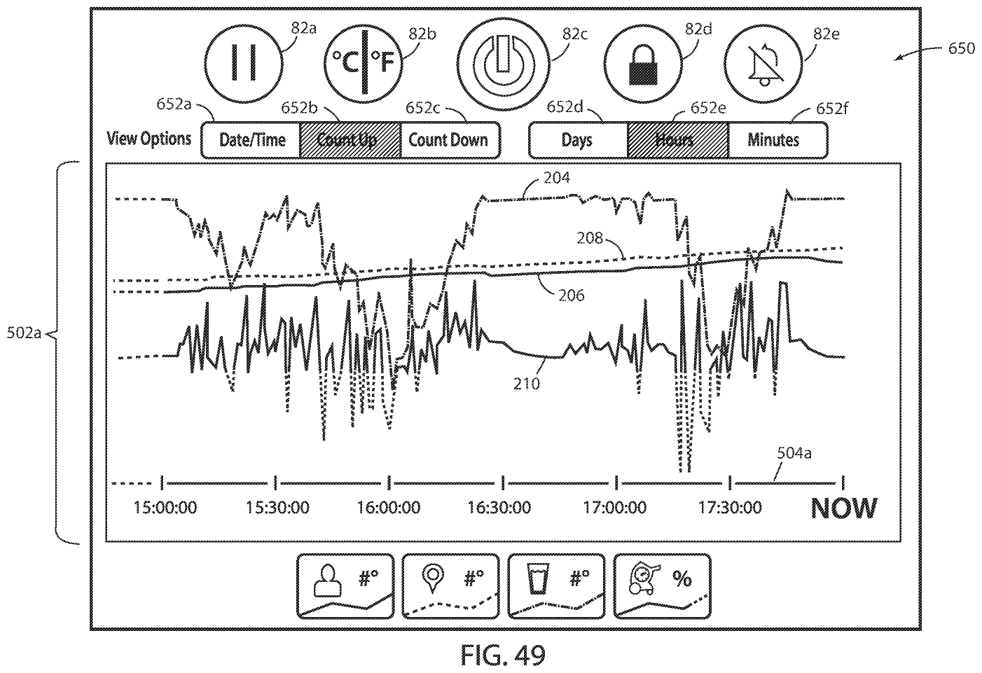

FIGS. 48 and 49 illustrate a partial view of control panel 76 when it is displaying a viewing option screen 650. Viewing option screen 650 includes a graph 502a that is modified from the graph 502 of FIG. 45. Modified graph 502a differs from graph 502 in that it is not broken up into segments 506, but instead includes all of its parameters in a single location. As with graph 502, these parameters include a water temperature plot 204, a patient temperature plot 206, a target patient temperature plot 208, and a machine effort plot 210 (which, may be multi-colored to distinguish heating efforts from cooling efforts). Viewing option screen 650 further includes a plurality of viewing option selectors 652a-f. These viewing option selectors 652 include a data/time selector 652a, a count up selector 652b, a count down selector 652c, a days selector 652d, an hours selector 652e, and a minutes selector 652f. Selectors 652 allows the user to select different options for the manner in which data is displayed on graph 502a.

If a user selects date/time selector 652a, controller 60 is configured to add the date and time to the time axis 504a of graph 502a. In the examples illustrated in FIGS. 48 and 49, this is not shown. However, were the user to activate the date and time selector 652a, controller 60 would add the corresponding date and time to each of the labeled time increments along time axis 504a. If a user selects the count up selector 652b, controller 60 displays the time on time axis 504a in ascending numerical order from the moment the thermal therapy session started. In other words, the labeled time increments each indicate how much time has elapsed since therapy began. An example of this selection is illustrated in FIG. 49, where the time increments along time axis 504a increase from left to right until the present moment (“NOW”).

If a user selects the count down selector 652c, controller 60 is configured to display the time on time axis 504 in descending numerical order from the time the therapy began until the present moment (“NOW”). An example of this selection is shown in FIG. 48, where the time increments along time axis 504a decrease from left to right and reach zero at the present moment. If the user selects the days selector 652d, controller 60 displays the units of time along time axis 504a in days (not shown). If the user selects the hours selector 652e, controller 60 displays the units of time along time axis 504a in hours, such as is shown in FIG. 49. If the user selects the minutes selector 652f, controller 60 displays the units of time along time axis 504a in minutes, such as is shown in FIG. 48.

In some embodiments, controller 60 may be configured to make additional changes to the graph 502a displayed on screen 650 in response to a user selecting one of the time unit selectors 652d, 652e, and/or 652f. In such embodiments, controller 60 may, in addition to changing the units of time displayed along time axis 504a, also change the window of time displayed on graph 502a (i.e. the total amount of time displayed on graph 502a). For example, when the user selects the days selector 652d, controller 60 may be configured to display several days worth of data on graph 502a. When the user selects the hours selector 652e, controller 60 may be configured to display only a several hours (or any number of hours less than or equal to 24 hours) worth of data on graph 502a. And when the user selects the minutes selector 652f, controller 60 may be configured to display sixty minutes, or less, worth of data on graph 502a. Of course, other time periods worth of data may be selected in response to the selection of these selectors 652d-f.

Although not shown in the drawings, controller 60 is also configured to display a time window selection control that allows a user to change the amount of time that is displayed on graph 502a (and/or any of the graphs described herein). This time window selection control works independently of the time selectors 652d-f described above. That is, this time window selection control will override the amount of time displayed as a result of the user selecting one of time selectors 652d-f. The user is thereby free to pick the precise amount of total time displayed on graph 502a, or any of the other graphs described herein. In some embodiments, the user can change the amount of time shown on graph 502a (and other graphs) by touching the graph with two fingers, and then either moving their fingers farther apart (to show more time) or pinching their fingers closer together (to show less time). Still other manners of selecting the window of time shown on graph 502a (and the other graphs described herein) may be used.

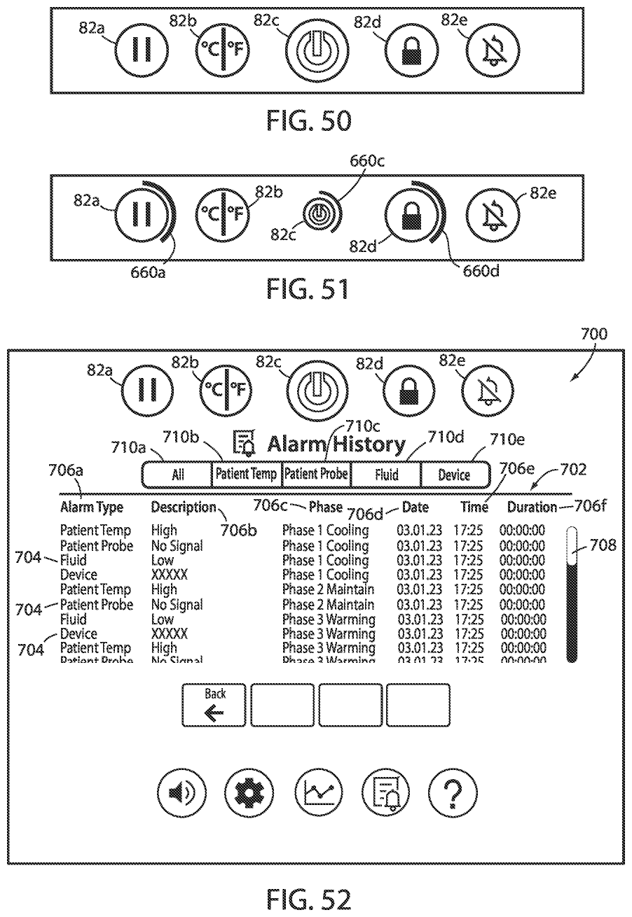

FIGS. 50 and 51 illustrate a plurality of controls 82a-e that are displayed on many of the screens discussed herein. In some embodiments, controller 60 is configured to display power control 82c in a first manner when no thermal therapy session is in progress, and in a second and different manner when a thermal therapy session is in progress. One example of this differential display of power control 82c is shown in FIGS. 50 and 51. In FIG. 51, power control 82c is displayed in a manner corresponding to no thermal therapy session being in progress. In FIG. 52, power control 82c is displayed in a manner corresponding to a thermal therapy session being in progress. As can be seen, controller 60 has reduced the size of power control 82c in FIG. 51 (when therapy is in progress) relative to the size of power control 82c in FIG. 50 (when no therapy is in progress).

In some embodiments of thermal control unit 22, controller 60 may also be configured to change a color in which power control 82c is displayed, depending upon whether therapy is currently in progress or not. In such embodiments, controller 60 may, for example, display power control 82c with a green shade when no therapy is in progress and a with a red, amber, or gray shade when therapy is in progress. In general, controller 60 may be configured to change the display of power control 82c to a less prominent display during thermal therapy sessions because it is unusual for a user to wish to turn off thermal control unit 22 during a thermal therapy session. In other embodiments, controller 60 may be configured to change the color of power control 82c to indicate different states of thermal control unit 22, such as display power control 82c in a green shade when power is not currently turned on (in which case touching power control 82c will turn on thermal control unit 22), a gray shade when power is currently supplied (in which case touching power control 82c will turn off thermal control unit 22), and a yellow or amber shade when power has been lost (e.g. a power failure occurs, the power plug for thermal control unit 22 gets unplugged, etc.).

In some embodiments of thermal control unit 22, controller 60 is configured to require the user to press and hold certain controls 82 before implementing their corresponding function. This feature helps prevent the user from accidentally implementing functions that may be particularly undesirable if implemented at an unintended time. The particular controls 82 that have this touch-and-hold feature may vary from embodiment to embodiment. In one embodiment, controller 60 is configured to implement this touch-and-hold feature for pause control 82a, power control 82c, and lock control 82d. In this particular embodiment, if the user wishes to use any of these controls 82a, 82c, and/or 82d, he or she needs to press their finger against these controls and hold their finger against the control for a short period of time (e.g. about one to two seconds, although other time periods may, of course be used).

In some of the embodiments of thermal control unit 22 that have the press-and-hold feature, when a user presses on controls 82a, 82c, and/or 82d, controller 60 is configured to start displaying a portion of a circle around the corresponding control 82a, 82c, and 82d that gets larger and larger until the circle is completely illustrated, at which point the corresponding control 82a, 82c, and/or 82d is implemented. An example of this is shown in FIG. 51 for each of controls 82a, 82c, and 82d. As shown therein, a partial circle 660a, 660b, and 660c is shown around each of these controls 82a, 82c, and 82d, respectively. As was mentioned previously, each partial circle 660 will continue to enlarge, as long as the user continues to press against the corresponding control 82, until the partial circle 660 becomes a full circle, at which point the function corresponding to that particular control 82 is implemented. If the user lifts his or her finger prior to the partial circle 660 becoming a full circle, controller 60 removes the partial circle 660 and the user—to the extent they still want to implement that particular function-needs to press down on the control again and hold it (and the partial circle 660 will then start over again at its smallest size).

Although FIG. 51 illustrates all three of controls 82a, 82c, and 82d as having partial circles 660, it will be understood that typically only a single one of controls 82a, 82c, or 82d will be pressed at any given moment, in which case only an ever-expanding partial circle 660 will be displayed adjacent to that particular control.

FIG. 52 illustrates a partial view of control panel 76 when it is displaying an alarm history screen 700. Alarm history screen 700 includes an alarm table 702 that includes a plurality of individual alarms 704 in the rows of table 702 and a plurality of alarm characteristics 706 in the columns of alarm table 702. The alarm characteristics 706 include an alarm type 706a, and alarm description 706b, an alarm phase 706c, an alarm date 706d, an alarm time 706e, and an alarm duration 706f. The alarm type 706a indicates the type of alarm, such as a patient temperature alarm (e.g. the patient's temperature goes outside of a specified range); a patient probe alarm (e.g. the patient temperature sensor 86 becomes disconnected, reports erroneous values, etc.); a fluid alarm (e.g. low fluid, too much fluid, etc.); or a device alarm (e.g. an alarm relating to other aspects of thermal control unit 22). The alarm description 706b provides more information about the condition that caused the alarm. The alarm phase 706c indicates at what phase of the thermal therapy session the alarm took place. The date 706d indicates the date on which the alarm occurred. The time 706e indicates the time at which the alarm occurred, and the duration 706f indicates how long the alarm lasted until it was rectified.

In some embodiments, controller 60 is configured to display a scroll bar 708 on alarm history screen 700. Scroll bar 708 allows the user to scroll up or down through the entries in table 702. In some embodiments, controller 60 is configured to list the alarms 704 in table 702 chronologically from the top to the bottom with most recent at the top and the oldest at the bottom.

Alarm history screen 700 may also include a plurality of alarm filter controls 710 that, when selected, filter the alarms listed in table 702. In the particular embodiment illustrated in FIG. 52, alarm history screen 700 includes an “all” alarm filter control 710a, a patient temperature filter control 710b, a patient probe filter control 710c, a fluid filter control 710d, and a device filter control 710e. When the user selects the all alarm filter control 710a, controller 60 displays all of the alarms stored in thermal control unit 22 in table 702. When a user selects one of the other filter controls 710b, c, d, or e, controller 60 only displays in table 702 the alarms that match the corresponding filter control 710 that was selected. For example, if the user presses the patient probe alarm filter control 710c, controller 60 will only display alarms 704 in table 702 that are of the “patient probe” type. In some embodiments, controller 60 is configured to allow the user to select multiple ones of controls 710b, c, e, and e at once, thereby allowing the user to filter the alarms 704 according to multiple categories simultaneously. Subsequently pressing the “all” control 710 erases all of the previously implemented filters and, as noted, causes controller 60 to display all of the stored alarms.

It will be understood that any of the screens and/or features shown in FIGS. 1-52 herein are merely representative of the types of screens and/or features that system 20 may be configured to display. Additional screens and/or modified screens may be included for carrying out any of the features and functions described herein. It will also be understood that any of the screens and/or functionality illustrated in FIGS. 45-52 herein may be alternatively displayed on a screen shown on a user device 170, rather than, or in addition to, being displayed on control panel 76 of thermal control unit 22. As a result, any of the above described functions that a user was able to carry out via control panel 76 of thermal control unit 22 may be carried out using a user device 170.

It will also be understood that thermal control system 20 may be combined with one or more other systems that are executed by one or more servers within a healthcare facility and that assist the caregivers in that facility. For example, in one embodiments, thermal control system 20 is modified to include any and/or all of the functionality of the caregiver assistance system disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which has already been incorporated herein by reference. When thermal control system is combined with such a caregiver assistance system, user devices 170 are capable of not only displaying the information discussed herein and implementing the features and functions discussed herein, but they are also able to display and/or carry out any of the same features and/or functions of the portable electronic devices 104 disclosed in the aforementioned PCT/US2020/039587 patent application.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1-18. (canceled)

19. A thermal control unit for controlling a patient's temperature during a thermal therapy session, the thermal control unit comprising:
(a) a circulation channel coupled to a fluid inlet and a fluid outlet;
(b) a pump for circulating fluid through the circulation channel from the fluid inlet to the fluid outlet;
(c) a heat exchanger adapted to add or remove heat from the fluid circulating in the circulation channel;
(d) a fluid temperature sensor adapted to detect a temperature of the fluid;
(e) a patient temperature probe port adapted to receive patient temperature readings from a patient temperature probe;
(f) a control panel comprising a display adapted to display data and a control thereon; and
(g) a controller adapted to control the heat exchanger in response to readings from the fluid temperature sensor and the patient temperature probe in order to control the patient's temperature, to display data on the display, and to implement a function in response to a user activating the control.

20. The thermal control unit of claim 19 wherein the controller is adapted to display a graph of thermal data over a period of time, a movable cursor adapted to be moved to a user-selectable moment in time, and a set of information corresponding to the user-selected moment in time, wherein the controller is further configured to update the set of information as the user moves the movable cursor.

21. The thermal control unit of claim 20 wherein the controller is further configured to display a horizontal time axis and a vertical bar attached to the movable cursor, and wherein the moment in time corresponds to a time defined by an intersection of the vertical bar with the horizontal time axis.

22. The thermal control unit of claim 20 wherein the set of information is displayed inside of an oval shape and the set of information includes a patient temperature, a fluid temperature, a patient target temperature, and a machine effort.

23. (canceled)

24. The thermal control unit of claim 19 wherein the controller is further adapted to display a time axis and a plurality of event icons associated with events, wherein the event icons are displayed along the time axis at locations corresponding to respective times at which the events occurred.

25. The thermal control unit of claim 24 wherein the controller is further adapted to display a skip forward control and, in response to a user activating the skip forward control, to display data corresponding to a next most recent event associated with one of the plurality of event icons.

26. (canceled)

27. The thermal control unit of claim 24 wherein the controller is further adapted to automatically add display an event icon in response to detecting an event, wherein the event is at least one of the following: (a) a patient temperature reading deviating from a target patient temperature by more than a threshold; (b) a malfunction associated with the patient temperature probe port; (c) a change in a phase of the thermal therapy session; (d) a pause in the thermal therapy session; or (e) an alarm.

28. (canceled)

29. The thermal control unit of claim 19 wherein the controller is further adapted to display a graph of a desired patient temperature over a period of time and a range in which the patient's temperature is desirably maintained, wherein the range is displayed on the graph as an upper line positioned above the desired patient temperature and a lower line positioned below the desired patient temperature, wherein both the upper line and the lower line are graphed over the period of time, and wherein the upper line and lower line are spaced apart from each other by an amount corresponding to the range in which the patient's temperature is desirably maintained.

30-31. (canceled)

32. The thermal control unit of claim 19 wherein the controller is further adapted display a preset therapy selector that allows a user to select a preset therapy; wherein the preset therapy includes definitions for a plurality of phases of the thermal therapy session; wherein the controller is configured to follow the definitions for the plurality of phases when following the preset therapy; the plurality of phases include a cooling phase, a maintenance phase, and a warming phase; and wherein the controller is further adapted to display a preset summary indicating information about each of the plurality of phases of the preset therapy, the preset summary including a phase indicator for each of the plurality of phases of the preset therapy, and wherein each phase indicator includes an icon identifying a type of phase and text describing the phase of each phase indicator.

33-35. (canceled)

36. The thermal control unit of claim 32 wherein both the icon and the text are positioned inside of an oval displayed on the display, each phase indicator is arranged vertically above another phase indicator in chronological order starting with a first phase indicator on a top of the preset summary and a second phase indicator below the first phase indicator, and wherein the controller is configured to fill in more and more of the oval as the thermal therapy session progresses through the phase corresponding to the oval.

37-39. (canceled)

40. The thermal control unit of claim 19 wherein the controller is further adapted to display a graph of thermal data over a period of time, and wherein the controller is further adapted to allow the user to select a characteristic of the graph.

41. The thermal control unit of claim 40 wherein the characteristic is a unit of time, the controller is adapted to display the graph with a horizontal time axis in whichever unit of time the user selects, and the controller is adapted to allow the user to select one of the following units of time: days, hours, or minutes.

42. (canceled)

43. The thermal control unit of claim 40 wherein the characteristic is a first direction in which time measurements are displayed and a second direction in which time measurements are displayed, wherein the first direction includes increasing numerical measurements from a left side of the display to a right side of the display such that the numerical measurements are measurements of a total amount of time elapsed since the thermal therapy session began, and wherein the second direction includes increasing numerical measurements from the right side of the display to the left side of the display such that the numerical measurements are measurements of a total amount of time prior to the present time.

44-45. (canceled)

46. The thermal control unit of claim 19 wherein the controller is adapted to display a power control on the display and to change a characteristic of the power control when the thermal control unit is implementing the thermal therapy session when compared to when the thermal control unit is not implementing the thermal therapy session, wherein the characteristic is one of a size of the power control or a color of the power control.

47-49. (canceled)

50. The thermal control unit of claim 19 wherein the controller is adapted to display a power control on the display, and wherein the controller is further adapted to change a color of the power control when the thermal control unit is in different states, wherein the different states include a first state in which the power control has not been activated to turn on power to the thermal control unit, a second state in which the power control has been activated to turn on power to the thermal control unit, and a third state in which electrical power is no longer supplied from a mains electrical outlet to the thermal control unit.

51-52. (canceled)

53. The thermal control unit of claim 50 wherein the controller is configured to display a plot of an amount of effort exerted by the thermal control unit over time, and to display the plot in a first color for moments in time when the thermal control unit is heating the circulating fluid and in a second color for moments in time when the thermal control unit is cooling the circulating fluid.

54-56. (canceled)

57. The thermal control unit of claim 19 wherein the controller is adapted to implement the function in response to the user activating the control if a user presses and holds the control for longer than a predetermined amount of time, but to not implement the function if the user presses the control for less than the predetermined amount of time, wherein the predetermined amount of time falls within a range of a half a second to three seconds, and wherein the control is one of a power control, a pause control, or a lock control.

58-59. (canceled)

60. The thermal control unit of claim 19 further comprising a transceiver adapted to communicate with a server, and wherein the controller is further adapted to display a first icon on the display indicating connectivity of the transceiver to a local area network of a facility in which the thermal control unit is located.

61. The thermal control unit of claim 60 wherein the controller is further adapted to display a second icon on the display indicating connectivity of the transceiver to the server, a third icon on the display indicating a software update is available for the thermal control unit, and a fourth icon on the display indicating information shown on the display may be displayed in different languages.

62-66. (canceled)

67. The thermal control unit of claim 19 wherein the controller is further adapted to display an alarm history screen and an alarm filter, the alarm history screen including a listing of alarms that were active during the thermal therapy session, the alarm filter adapted to filter the alarm listing according to a type of alarm selected by the user, and wherein, in response to a user activating the alarm filter, the controller is configured to only include in the alarm listing those alarms matching the type of alarm selected by the user, wherein the type of alarm selected by the user includes at least one of the following: a patient temperature alarm, a patient temperature sensor alarm, a fluid alarm, or a device alarm.

68-71. (canceled)
